SEQUENCE LISTING (17 sequences)

SEQ ID NO: 1 - DNA, 34 bp, Artificial Sequence
  Oligonucleotide Primer spnKF
gggaattccatatgtccacaacgcacgagatcga

SEQ ID NO: 2 - DNA, 34 bp, Artificial Sequence
  Oligonucleotide Primer spnKR
gccgctcgagctcgtcctccgcgctgttcacgtc

SEQ ID NO: 3 - DNA, 23 bp, Artificial Sequence
  Oligonucleotide Primer Forward Primer #1
cggtgcccgaattccatgacccg

SEQ ID NO: 4 - DNA, 33 bp, Artificial Sequence
  Oligonucleotide Primer Reverse Primer #1
gtgcgttctagacatatgagctcctcatggctg

SEQ ID NO: 5 - DNA, 33 bp, Artificial Sequence
  Oligonucleotide Primer Forward Primer #2
gtgccatctagactggacgacatattgcacctg

SEQ ID NO: 6 - DNA, 31 bp, Artificial Sequence
  Oligonucleotide PrimerReverse Primer #2
gaatgcgaagcttacgatctcgtcgtccgtg

SEQ ID NO: 7 - DNA, 24 bp, Artificial Sequence
  Oligonucleotide Primer spnK Del Validation 1 Forward
gttcacggtgattccggtgactcg

SEQ ID NO: 8 - DNA, 24 bp, Artificial Sequence
  Oligonucleotide PrimerspnK Del Validation 1 Reverse
acctgcactgcttcctggagcttc

SEQ ID NO: 9 - DNA, 60 bp, Artificial Sequence
  spnK sequence containing an internal deletion
atgtctagactggacgacatattgcacctggccgacgtgaacagcgcggaggacgagtga

SEQ ID NO: 10 - DNA, 1147 bp, Artificial Sequence
  spnK sequence containing a 5′ end deletion
ggagctcatcacggcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgagttccgtgatcgcccggtgcgcatcctggagatcggtgtcggtggctacaacttcgacggtggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagtacggaccgttcgacatcatcatcgacgatggcagccacatcaacggacacgtgcgcacatccctggaaacgctgttcccccggttgcgcagcggtggcgtatacgtgatcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacatcgcgttcctggagaaaggcgtcaacgccgaaggcggcgttcctgcttgggtgccaaggagtctggacgacatattgcacctggccgacgtgaacagcgcggaggacgagtga

SEQ ID NO: 11 - DNA, 1134 bp, Artificial Sequence
  Deletion of the first putative S-adenosyl- methionine-dependent methyltransferase domain of spnK
atgtccacaacgcacgagatcgaaaccgtggaacgcatcatcctcgccgccggatccagtgcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgatggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagtacggaccgttcgacatcatcatcgacgatggcagccacatcaacggacacgtgcgcacatccctggaaacgctgttcccccggttgcgcagcggtggcgtatacgtgatcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacatcgcgttcctggagaaaggcgtcaacgccgaaggcggcgttcctgcttgggtgccaaggagtctggacgacatattgcacctggccgacgtgaacagcgcggaggacgagtga

SEQ ID NO: 12 - DNA, 1084 bp, Artificial Sequence
  Deletion of second putative S-adenosyl- methionine-dependent methyltransferase domain of spnK
atgtccacaacgcacgagatcgaaaccgtggaacgcatcatcctcgccgccggatccagtgcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgagttccgtgatcgcccggtgcgcatcctggagatcggtgtcggtggctacaacttcgacggtggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacatcgcgttcctggagaaaggcgtcaacgccgaaggcggcgttcctgcttgggtgccaaggagtctggacgacatattgcacctggccgacgtgaacagcgcggaggacgagtga

SEQ ID NO: 13 - DNA, 1124 bp, Artificial Sequence
  spnK nucleotide sequence containing a 3′ end deletion
atgtccacaacgcacgagatcgaaaccgtggaacgcatcatcctcgccgccggatccagtgcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgagttccgtgatcgcccggtgcgcatcctggagatcggtgtcggtggctacaacttcgacggtggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagtacggaccgttcgacatcatcatcgacgatggcagccacatcaacggacacgtgcgcacatccctggaaacgctgttcccccggttgcgcagcggtggcgtatacgtgatcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacacagcagaggggcgaacacacaggcatttccgaccgcggatcag

SEQ ID NO: 14 - DNA, 1207 bp, Artificial Sequence
  spnK and adjoining upstream sequence
ggagctcatcacgatgtccacaacgcacgagatcgaaaccgtggaacgcatcatcctcgccgccggatccagtgcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgagttccgtgatcgcccggtgcgcatcctggagatcggtgtcggtggctacaacttcgacggtggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagtacggaccgttcgacatcatcatcgacgatggcagccacatcaacggacacgtgcgcacatccctggaaacgctgttcccccggttgcgcagcggtggcgtatacgtgatcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacatcgcgttcctggagaaaggcgtcaacgccgaaggcggcgttcctgcttgggtgccaaggagtctggacgacatattgcacctggccgacgtgaacagcgcggaggacgagtga

SEQ ID NO: 15 - DNA, 1238 bp, Artificial Sequence
  spnK and adjoining downstream sequence
atgtccacaacgcacgagatcgaaaccgtggaacgcatcatcctcgccgccggatccagtgcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgagttccgtgatcgcccggtgcgcatcctggagatcggtgtcggtggctacaacttcgacggtggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagtacggaccgttcgacatcatcatcgacgatggcagccacatcaacggacacgtgcgcacatccctggaaacgctgttcccccggttgcgcagcggtggcgtatacgtgatcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacatcgcgttcctggagaaaggcgtcaacgccgaaggcggcgttcctgcttgggtgccaaggagtctggacgacatattgcacctggccgacgtgaacagcgcggaggacgagtgaacagcagaggggcgaacacacaggcatttccgaccgcggatcag

SEQ ID NO: 16 - DNA, 70 bp, Saccharopolyspora spinosa
ccgccggcacggctttcacccggtcagccatgaggagctcatcacgatgtccacaacgcacgagatcgaa

SEQ ID NO: 17 - DNA, 1194 bp, Saccharopolyspora spinosa
atgtccacaacgcacgagatcgaaaccgtggaacgcatcatcctcgccgccggatccagtgcggcgagcctggccgacctgaccaccgaactcggactcgccaggatcgcacccgtgctgatcgacgagatcctcttccgcgcggaaccggcccccgacatcgaacggaccgaggtcgcggtccagatcacccaccgaggcgagaccgttgacttcgtcctgacgctacagtccggtgagctgatcaaggccgagcaacgaccggtcggagacgtcccgctgcggatcggttacgagctcaccgatctcatcgccgagttgttcggcccaggagctcccagggccgtcggcgcccggagcaccaacttcctccgaaccaccacatccggttcgatacccggtccgtcggaactgtccgatggcttccaggccatctccgcagtggtcgccggctgcgggcaccgacgtcccgacctcaacttgctcgcctcccactaccgcacggacaagtggggcggcctgcactggttcaccccgctatacgagcgacacctcggcgagttccgtgatcgcccggtgcgcatcctggagatcggtgtcggtggctacaacttcgacggtggcggcggcgaatccctgaagatgtggaagcgctacttccaccgcggcctcgtgttcgggatggacgttttcgacaagtccttcctcgaccagcagaggctctgcaccgtccgcgccgaccagagcaagcccgaggagctggccgccgttgacgacaagtacggaccgttcgacatcatcatcgacgatggcagccacatcaacggacacgtgcgcacatccctggaaacgctgttcccccggttgcgcagcggtggcgtatacgtgatcgaggatctgtggacgacctatgctcccggattcggcgggcaggcgcagtgcccggccgcacccggcaccacggtcagcctgctcaagaacctgttggaaggcgttcagcacgaggagcagccgcatgcgggctcgtacgagccgagctacctggaacgcaatttggtcggcctccacacctaccacaacatcgcgttcctggagaaaggcgtcaacgccgaaggcggcgttcctgcttgggtgccaaggagtctggacgacatattgcacctggccgacgtgaacagcgcggaggacgagtga

(19) United States
(12) Patent Application Publication
(10) Pub. No.: US 2011/0281359 A1
(43) Pub. Date: Nov. 17, 2011

(54) SPNK STRAINS

(75) Inventor: Lei Han, Carmel, IN (US)

(73) Assignee: DOW AGROSCIENCES LLC, Indianapolis, IN (US)

(21) Appl. No.: 13/105,241

(22) Filed: May 11, 2011

Related U.S. Application Data
(60) Provisional application No. 61/333,540, filed on May 11, 2010.

(51) Int. Cl. C12N 15/01; C12N 15/00; C12N 1/00; C12N 15/09

(52) U.S. Cl. 435/441; 435/471; 435/440; 435/243

(57) ABSTRACT

This invention includes spinosyn biosynthetic genes, spinosyn producing microorganisms transformed with the biosynthetic genes, methods using the biosynthetic genes to increase production of spinosyn insecticidal macrolides, and methods using the genes or fragments thereof to change the products produced by spinosyn-producing microorganisms. Additionally, the present invention includes methods and compositions for converting a spinosyn A and D producing strain to a spinetoram precursor, spinosyn J and L, producing strain.

### CROSS-REFERENCE TO RELATED APPLICATION

This patent application claims the benefit of U.S. Provisional Patent Application No. 61/333,540, filed May 11, 2010, which is incorporated herein by reference in its entirety.

### FIELD OF THE INVENTION

The invention applies the technical field of molecular genetics to disrupt the expression of genes. More specifically, it has been discovered that a mutation in the spnK gene converts a spinosad producing strain to a spinetoram precursor producing strain.

### BACKGROUND

As disclosed in U.S. Pat. No. 5,362,634, fermentation product A83543 is a family of related compounds produced by Saccharopolyspora spinosa. The known members of this family have been referred to as factors or components, and each has been given an identifying letter designation. These compounds are hereinafter referred to as spinosyn A, B, etc. The spinosyn compounds are useful for the control of arachnids, nematodes and insects, in particular, Lepidoptera and Diptera species, and they are quite environmentally friendly and have an appealing toxicological profile.

The naturally produced spinosyn compounds consist of a 5,6,5-tricylic ring system, fused to a 12-membered macrocyclic lactone, a neutral sugar (rhamnose) and an amino sugar (forosamine) (see Kirst et al. (1991). If the amino sugar is not present the compounds have been referred to as the pseudoaglycone of A, D, etc., and if the neutral sugar is not present then the compounds have been referred to as the reverse pseudoaglycone of A, D, etc. A more preferred nomenclature is to refer to the pseudoaglycones as spinosyn A 17-Psa, spinosyn D 17-Psa, etc., and to the reverse pseudoaglycones as spinosyn A 9-Psa, spinosyn D 9-Psa, etc.

The naturally produced spinosyn compounds may be produced via fermentation from cultures NRRL 18395, 18537, 18538, 18539, 18719, 18720, 18743 and 18823. These cultures have been deposited and made part of the stock culture collection of the Midwest Area Northern Regional Research Center, Agricultural Research Service, United States Department of Agriculture, 1815 North University Street, Peoria, Ill., 61604.

U.S. Pat. No. 5,362,634 and corresponding European Patent Application No. 375316 A1 relate to spinosyns A, B, C, D, E, F, G, H, and J. These compounds are said to be produced by culturing a strain of the novel microorganism Saccharopolyspora spinosa selected from NRRL 18395, NRRL 18537, NRRL 18538, and NRRL 18539.

WO 93/09126 relates to spinosyns L, M, N, Q, R, S, and T. Also discussed therein are two spinosyn J producing strains: NRRL 18719 and NRRL 18720, and a strain that produces spinosyns Q, R, S, and T: NRRL 18823.

WO 94/20518 and U.S. Pat. No. 5,6704,486 relates to spinosyns K, O, P, U, V, W, and Y, and derivatives thereof. Also discussed is spinosyn K-producing strain NRRL 18743.

A challenge in producing spinosyn compounds arises from the fact that a very large fermentation volume is required to produce a very small quantity of spinosyns. It is highly desired to increase spinosyn production efficiency and thereby increase availability of the spinosyns while reducing their cost.

It would also be advantageous to provide cloned biosynthetic genes that provide a method for producing new derivatives of the spinosyns which may have a different spectrum of insecticidal activity. New derivatives are desirable because, although known spinosyns inhibit a broad spectrum of insects, they do not control all pests. Different patterns of control may be provided by biosynthetic intermediates of the spinosyns, or by their derivatives produced in vivo, or by derivatives resulting from their chemical modification in vitro.

It would also be advantageous to provide novel intermediates synthesized by mutant strains of S. spinosa in which parts of certain genes encoding enzymes for spinosyn biosynthesis have been replaced with parts of the same gene which have been specifically mutated in vitro, or with corresponding parts of genes from other organisms.

### SUMMARY OF THE INVENTION

The present invention provides processes for converting a spinosad producing strain, such as spinosyn A and D, to a spinetoram precursor producing strain, such as spinosyn J and L. Such process can include the production of a modification in the spnK gene to eliminate 3′-O-methyltransferase activity. The modification can be made through in-frame deletions, mutations, substitutions, deletions, insertions and the like. The in-frame deletions can be throughout the gene include deletions of the 5′ end, the 3′ end, or of a spnK coding region. One such in-frame deletion can include SEQ. ID. NO. 9. Point mutations can include, but are not limited to, mutations at locations base pair 528, 589, 602, 668, 721, 794, 862, 895, 908, 937 and 1131. These mutations can lead to changes in the translation of the spnK gene. Such changes can be amino acid changes, substitutions, or the creation of stop codons. Such modifications result in the spinosyn compound production of spinosyn J and L as compared to spinosyn A and D.

Particular methods of the present invention include the conversion of a spinosad producing strain to a spinetoram precursor producing strain by disabling a spnK gene while maintaining spinosyn J and L production. The disabling or disruption of normal spnK protein activity can occur by in-frame deletions, mutations, substitutions, deletions, insertions and the like. It can also be caused by manipulations to the promoter or the ribosome binding site sequences.

The invention further provides a genetically modified host cell that produces a spinetoram precursor. This genetically modified host can be produced by modifying the spnK gene to eliminate 3′-O-methyltransferase activity. The modification can be made through in-frame deletions, mutations, substitutions, deletions, insertions and the like. The in-frame deletions can include deletions of the 5′ end, the 3′ end, or of a spnK coding region.

The invention also provides processes for converting spinosad producing strains to spinetoram precursor producing strains by modifying the spnK gene to eliminate 3′-O-methyltransferase activity. This process can include in-frame deletions, point mutations, deletions, and insertions. Such in-frame deletions can include in-frame deletions of a 5′ end, in-frame deletions of a 3′ end, and in-frame deletions of a spnK coding region. The deletions can include a single or multiple nucleotide base deletions that disrupts the normal reading frame of the spnK gene. Insertions can include single or multiple nucleotide base insertions that disrupts the normal reading frame of the spnK gene. Point mutations can occurs in the base pair locations 528, 589, 602, 668, 721, 794, 862, 895, 908, 937 and 1131. These point mutations can result in amino acid substitutions in the active site or the substrate binding site of the spnK gene.

The invention also includes genetically modified host cells that produce a spinetoram precursor, wherein the genetically modified host cell is a prokaryotic host cell that does not normally produce significant amount of spinetoram precursor by producing a modification in the spnK gene to eliminate 3′-O-methyltransferase activity. Other embodiments include methods of converting a spinosad producing strain to a spinetoram precursor producing strain by disabling a spnK gene while maintaining spinosyn J and L production. Such methods can include in-frame deletions, point mutations, deletions, and insertions. Such in-frame deletions can include in-frame deletions of a 5′ end, in-frame deletions of a 3′ end, and in-frame deletions of a spnK coding region. The deletions can include a single or multiple nucleotide base deletions that disrupts the normal reading frame of the spnK gene. Insertions can include single or multiple nucleotide base insertions that disrupts the normal reading frame of the spnK gene. Point mutations can occurs in the base pair locations 528, 589, 602, 668, 721, 794, 862, 895, 908, 937 and 1131. These point mutations can result in amino acid substitutions in the active site or the substrate binding site of the spnK gene. Other methods of disabling the spnK may occur by manipulating a ribosome binding site or by manipulating a promoter of a spnK gene.

### BRIEF DESCRIPTION OF THE FIGURES

FIG. 1 illustrates the location of the spnK point mutations. The mutations are highlighted within the wild-type sequence of spnK (SEQ ID NO: 17).

FIG. 2 depicts a physical map of spnJ, spnK, spnL and spnM. The PCR products that were produced are indicated by the lines below the chromosome map.

FIG. 3 demonstrates the integration of the spnK in-frame deletion construct within the spnLM region as a single crossover homologous recombination according to an embodiment of the present invention. (Asterisk indicates incomplete coding sequence of spnJ and spnM).

FIG. 4 illustrates double crossover mutants which resulted in a deletion of the spnK gene according to an embodiment of the present invention. The size and DNA sequence of the PCR fragment indicates in-frame deletion of the spnK gene.

FIG. 5 is a diagram of the insertion cassette containing an in-frame apramycin resistance gene cassette (aac(3)IV) within spnK according to an embodiment of the present invention.

FIG. 6 depicts the ribosome binding site (labeled as Shine-Dalgarno) which is located upstream of the spnK coding sequence according to an embodiment of the present invention (SEQ ID NO:16). This sequence is highlighted in the figure.

### DETAILED DESCRIPTION OF THE INVENTION

There are many uses for the cloned Saccharopolyspora spinosa DNA. The cloned genes can be used to improve yields of spinosyns and to produce new spinosyns. Improved yields can be obtained by integrating into the genome of a particular strain a duplicate copy of the gene for whatever enzyme is rate limiting in that strain. In cases wherein the biosynthetic pathway is blocked in a particular mutant strain due to lack of a required enzyme, production of the desired spinosyns can be restored by integrating a copy of the required gene. Where a biosynthetic pathway is disrupted, a different precursor strain can be created. More specifically the disruption of the spnK gene can result in spinosyn J and L production as compared to spinosyn A and D production.

Novel spinosyns can be produced using fragments of the cloned DNA to disrupt steps in the biosynthesis of spinosyns. Such disruption may lead to the accumulation of precursors or “shunt” products (the naturally-processed derivatives of precursors). The fragments useful in carrying out disruptions are those internal to a gene with bases omitted from both the 5′ and 3′ ends of the gene as well as throughout the gene. Homologous recombination events utilizing such fragments result in two partial copies of the gene: one that is missing the omitted bases from the 5′ end and one that is missing the omitted bases from the 3′ end. The number of bases omitted at each end of the fragment must be large enough so that neither of the partial copies of the gene retains activity.

The following definitions are used herein and should be referred to for interpretation of the claims and the specification. Unless otherwise noted, all U.S. patents and U.S. patent applications referenced herein are incorporated by reference in their entirety.

As used herein, the indefinite articles “a” and “an” preceding an element or component of the invention are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

As used herein, the terms “comprising” and “including” mean the presence of the stated features, integers, steps, or components as referred to in the claims, but that it does not preclude the presence or addition of one or more other features, integers, steps, components or groups thereof. This means a composition, a mixture, a process, a method, an article, or an apparatus that “comprises” or “includes” a list of elements is not limited to only those elements but may include others not expressly listed or inherent to it. As used herein, “or” refers to an inclusive and an exclusive “or.” For example, a condition A or B is satisfied by any one of the following: A is true (or present) and B is false (or not present), A is false (or not present) and B is true (or present), and both A and B are true (or present).

As used herein, the term “about” refers to modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about,” the claims include equivalents to the quantities.

As used herein, the term “invention” or “present invention” is a non-limiting term and is intended to encompass all possible variations as described in the specification and recited in the claims.

As used herein, the terms “polypeptide” and “peptide” will be used interchangeably to refer to a polymer of two or more amino acids joined together by a peptide bond. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics. The peptides may comprise L-amino acids.

As used herein, the terms “peptide of interest,” “POI,” “gene product,” “target gene product,” and “target coding region gene product” refer to the desired heterologous peptide/protein product encoded by the recombinantly expressed foreign gene. The peptide of interest may include any peptide/protein product including, but not limited to proteins, fusion proteins, enzymes, peptides, polypeptides, and oligopeptides. The peptide of interest ranges in size from 2 to 398 amino acids in length.

As used herein, the term “genetic construct” refers to a series of contiguous nucleic acids useful for modulating the genotype or phenotype of an organism. Non-limiting examples of genetic constructs include but are not limited to a nucleic acid molecule, and open reading frame, a gene, an expression cassette, a vector, a plasmid and the like.

As used herein, the term “endogenous gene” refers to a native gene in its natural location in the genome of an organism.

As used herein, a “foreign gene” refers to a gene not normally found in the host organism, but that is introduced into the host organism by gene transfer. Foreign genes can comprise native genes inserted into a non-native organism, or chimeric genes.

As used herein, the term “heterologous” with respect to sequence within a particular organism/genome indicates that the sequence originates from a foreign species, or, if from the same species, is substantially modified from its native form in composition and/or genomic locus by deliberate human intervention. Thus, for example, heterologous gene expression refers to the process of expressing a gene from one organism/genome by placing it into the genome of a different organism/genome.

As used herein, the term “recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation, natural transduction, natural transposition) such as those occurring without deliberate human intervention.

The term “genetically engineered” or “genetically altered” means the scientific alteration of the structure of genetic material in a living organism. It involves the production and use of recombinant DNA. More in particular it is used to delineate the genetically engineered or modified organism from the naturally occurring organism. Genetic engineering may be done by a number of techniques known in the art, such as e.g. gene replacement, gene amplification, gene disruption, transfection, transformation using plasmids, viruses, or other vectors. A genetically modified organism, e.g. genetically modified microorganism, is also often referred to as a recombinant organism, e.g. recombinant microorganism.

As used herein, the term “disrupted” or “disruption” when referring to a gene that has been manipulated or modified through genetic engineering or through natural causes that change the activity of a gene. Such gene activity may be increased or decreased. Additionally, such disruption may abolish protein function. To facilitate such a decrease, the copy number of the genes may be decreased, such as for instance by underexpression or disruption of a gene. A gene is said to be “underexpressed” if the level of transcription of said gene is reduced in comparison to the wild type gene. This may be measured by for instance Northern blot analysis quantifying the amount of mRNA as an indication for gene expression. As used herein, a gene is underexpressed if the amount of generated mRNA is decreased by at least 1%, 2%, 5% 10%, 25%, 50%, 75%, 100%, 200% or even more than 500%, compared to the amount of mRNA generated from a wild-type gene. Alternatively, a weak promoter may be used to direct the expression of the polynucleotide. In another embodiment, the promoter, regulatory region and/or the ribosome binding site upstream of the gene can be altered to achieve the reduced expression. The expression may also be reduced by decreasing the relative half-life of the messenger RNA. In another embodiment, the activity of the polypeptide itself may be decreased by employing one or more mutations in the polypeptide amino acid sequence, which decrease the activity. For example, altering the affinity of the polypeptide for its corresponding substrate may result in reduced activity. Likewise, the relative half-life of the polypeptide may be decreased. In either scenario, that being reduced gene expression or reduced activity, the reduction may be achieved by altering the composition of the cell culture media and/or methods used for culturing. “Reduced expression” or “reduced activity” as used herein means a decrease of at least 5%, 10%, 25%, 50%, 75%, 100%, 200% or even more than 500%, compared to a wild-type protein, polynucleotide, gene; or the activity and/or the concentration of the protein present before the polynucleotides or polypeptides are reduced. The activity of the SpnK protein may also be reduced by contacting the protein with a specific or general inhibitor of its activity. The terms “reduced activity,” “decreased or abolished activity” are used interchangeably herein.

Expression “control sequences” refers collectively to promoter sequences, ribosome binding sites, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Recombination” refers to the reassortment of sections of DNA or RNA sequences between two DNA or RNA molecules. “Homologous recombination” occurs between two DNA molecules which hybridize by virtue of homologous or complementary nucleotide sequences present in each DNA molecule.

The terms “stringent conditions” or “hybridization under stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 Overview of principles of hybridization and the strategy of nucleic acid probe assays, Elsevier, N.Y. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al. (1989) Molecular Cloning—A Laboratory Manual (2nd ed.) Vol. 1-3, Cold Spring Harbor Laboratory, Cold Spring Harbor Press, NY, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

The invention also relates to an isolated polynucleotide hybridizable under stringent conditions, preferably under highly stringent conditions, to a polynucleotide as of the present invention.

As used herein, the term “hybridizing” is intended to describe conditions for hybridization and washing under which nucleotide sequences at least about 50%, at least about 60%, at least about 70%, more preferably at least about 80%, even more preferably at least about 85% to 90%, most preferably at least 95% homologous to each other typically remain hybridized to each other.

In one embodiment, a nucleic acid of the invention is at least 40%, 45%, 50%, 55%, 60%, 65%, 70%, 75%, 80%, 85%, 90%, 91%, 92%, 93%, 94%, 95%, 96%, 97%, 98%, 99%, or more homologous to a nucleic acid sequence shown in this application or the complement thereof.

Another non-limiting example of stringent hybridization conditions are hybridization in 6× sodium chloride/sodium citrate (SSC) at about 45° C., followed by one or more washes in 1×SSC, 0.1% SDS at 50° C., preferably at 55° C. more preferably at 60° C. and even more preferably at 65° C.

Highly stringent conditions can include incubations at 42° C. for a period of several days, such as 2-4 days, using a labeled DNA probe, such as a digoxigenin (DIG)-labeled DNA probe, followed by one or more washes in 2×SSC, 0.1% SDS at room temperature and one or more washes in 0.5×SSC, 0.1% SDS or 0.1×SSC, 0.1% SDS at 65-68° C. In particular, highly stringent conditions include, for example, 2 h to 4 days incubation at 42° C. using a DIG-labeled DNA probe (prepared by e.g. using a DIG labeling system; Roche Diagnostics GmbH, 68298 Mannheim, Germany) in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 minutes in 2×SSC and 0.1% SDS at room temperature and then washing twice for 15-30 minutes in 0.5×SSC and 0.1% SDS or 0.1×SSC and 0.1% SDS at 65-68° C.

In some embodiments an isolated nucleic acid molecule of the invention that hybridizes under highly stringent conditions to a nucleotide sequence of the invention can correspond to a naturally-occurring nucleic acid molecule. As used herein, a “naturally-occurring” nucleic acid molecule refers to an RNA or DNA molecule having a nucleotide sequence that occurs in nature (e.g., encodes a natural protein).

A skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

A cloned fragment of DNA containing genes for spinosyn biosynthetic enzymes would enable duplication of genes coding for rate limiting enzymes in the production of spinosyns. This could be used to increase yield in any circumstance when one of the encoded activities limited synthesis of the desired spinosyn. A yield increase of this type was achieved in fermentations of Streptomyces fradiae by duplicating the gene encoding a rate-limiting methyltransferase that converts macrocin to tylosin (Baltz et al., 1997).

Specific intermediates (or their natural derivatives) could be synthesized by mutant strains of S. spinosa in which certain genes encoding enzymes for spinosyn biosynthesis have been disrupted. Such strains can be generated by integrating, via homologous recombination, a mutagenic plasmid containing an internal fragment of the target gene. Upon plasmid integration, two incomplete copies of the biosynthetic gene are formed, thereby eliminating the enzymatic function it encoded. The substrate for this enzyme, or some natural derivative thereof, should accumulate upon fermentation of the mutant strain. Such a strategy was used effectively to generate a strain of Saccharopolyspora erythraea producing novel 6-deoxyerythromycin derivatives (Weber & McAlpine, 1992).

Such strains could be generated by swapping the target region, via double crossover homologous recombination, with a mutagenic plasmid containing the new fragment between non-mutated sequences which flank the target region. The hybrid gene would produce protein with altered functions, either lacking an activity or performing a novel enzymatic transformation. A new derivative would accumulate upon fermentation of the mutant strain. Such a strategy was used to generate a strain of Saccharopolyspora erythraea producing a novel anhydroerythromycin derivative (Donadio et al., 1993).

Spinosyn biosynthetic genes and related ORFs were cloned and the DNA sequence of each was determined. The cloned genes and ORFs are designated hereinafter as spnA, spnB, spnC, spnD, spnE, spnF, spnG, spnH, spnI, spnJ, spnK, spnL, spnM, spnN, spnO, spnP, spnQ, spnR, spnS, ORFL15, ORFL16, ORFR1, ORFR2, S. spinosa gtt, S. spinosa gdh, S. spinosa epi, and S. spinosa kre.

Saccharapolyspora spinosa produces a mixture of nine closely related compounds collectively called “spinosyns.” Within the mixture, spinosyn A and D, known as spinosad, are the major components and have the highest activity against key insect targets. Spinosyn J and L, two of the minor components within the spinosyn mixture, are the precursors for spinetoram, the second generation spinosyn insecticide. Embodiments of the invention concerns direct conversion of a spinosad producing strain to a spinetoram precursor producing strain via manipulations of spnK which encodes for 3′-O-methyltransferase.

Spinosad is an insecticide produced by Dow AgroSciences (Indianapolis, Ind.) that is comprised mainly of approximately 85% spinosyn A and approximately 15% spinosyn D. Spinosyn A and D are natural products produced by fermentation of Saccharopolyspora spinosa, as disclosed in U.S. Pat. No. 5,362,634. Spinosad is an active ingredient of several insecticidal formulations available commercially from Dow AgroSciences, including the TRACER™, SUCCESS™, SPINTOR™, and CONSERVE™ insect control products. For example, the TRACER product is comprised of about 44% to about 48% spinosad (w/v), or about 4 pounds of spinosad per gallon. Spinosyn compounds in granular and liquid formulations have established utility for the control of arachnids, nematodes, and insects, in particular Lepidoptera, Thysanoptera, and Diptera species. Spinosyn A and D is also referred to herein as Spinosyn A/D.

Spinetoram is a mixture of 5,6-dihydro-3′-ethoxy spinosyn J (major component) and 3′-ethoxy spinosyn L produced by Dow AgroSciences. The mixture can be prepared by ethoxylating a mixture of spinosyn J and spinosyn L, followed by hydrogenation. The 5,6 double bond of spinosyn J and its 3′-ethoxy is hydrogenated much more readily than that of spinosyn L and its 3′-ethoxy derivative, due to steric hindrance by the methyl group at C-5 in spinosyn L and its 3′-ethoxy derivative. See, U.S. Pat. No. 6,001,981. Spinosyn J and L is also referred to herein as Spinosyn J/L.

It has recently been demonstrated that spnK encodes for 3′-O-methyltransferase. See, Kim et al., JACS, 132(9): 2901-3 (2010). Applicants have found that spnK can be removed from the spinosyn biosynthetic gene cluster via in-frame double crossover homologous recombination without having a polar effect on the transcription of the downstream genes spnL and spnM. This allows for a spinosad producing strain to be engineered to produce a spinetoram precursor producing strain. This also indicates that the spnK knockout strain had lost the 3′-O-methyltransferase activity.

Embodiments of the present invention can include manipulations in the spnK gene that result in an in-frame deletion of the spnK gene by removing one or multiple codons in a spinosad producing strain. An in-frame deletion of the spnK gene can include any truncation of any part of the spnK gene. In-frame deletions according to the present invention include deletions which remove a segment of the protein coding sequence, yet retain the proper reading frame after the deletion. Some embodiments of the present invention can include deletions that are “clean deletions,” i.e., they contain no exogenous DNA sequences inserted into the gene or. An in-frame deletion of the spnK gene may include removal of anywhere from 1 to 397 amino acids. It can include removal of the start codon. It can further include removal of any conserved domain or any transcription initiation region.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

Embodiments of the present invention can include manipulations in the spnK gene that result in an in-frame deletion of the 5′ end of the spnK gene by removing one or multiple codons in a spinosad producing strain. These codons could include the first, second or third instance of an ATG codon.

Additional embodiments of the present invention can include manipulations in the spnK gene that result in an in-frame deletion of the 3′ end of the spnK gene by removing one or multiple codons in a spinosad producing strain.

Other embodiments of the present invention can include manipulations in the spnK gene in an in-frame deletion of the spnK coding region, either a single codon or multiple codons, while leaving both the 5′ end and 3′ end of the gene intact.

Additional embodiments of the present invention can include manipulations in the spnK gene that include single or multiple point mutations that result in premature transcription termination or amino acid substitution(s) in multiple sites, including, but not limited to, the active site and/or in the substrate binding site. Such single or multiple point mutations may occur within the SAM-binding motif, result in early termination be in the active site or the substrate binding site. Such single or multiple point mutations can also be in a location which affects the overall SpnK structure or affect proper folding which could abolish the SpnK function. Such single or multiple point mutations may be created through detection of functional polymorphisms or by mutagenesis.

“Functional polymorphism” as used herein refers to a change in the base pair sequence of a gene that produces a qualitative or quantitative change in the activity of the protein encoded by that gene (e.g., a change in specificity of activity; a change in level of activity). The term “functional polymorphism” includes mutations, deletions and insertions.

In general, the step of detecting the polymorphism of interest may be carried out by collecting a biological sample containing DNA from the source, and then determining the presence or absence of DNA containing the polymorphism of interest in the biological sample.

Determining the presence or absence of DNA encoding a particular mutation may be carried out with an oligonucleotide probe labeled with a suitable detectable group, and/or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the particular mutation. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al.

Amplification of a selected, or target, nucleic acid sequence may be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Qβ replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is generally preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. Such probes may be from 5 to 500 nucleotides in length, preferably 5 to 250, more preferably 5 to 100 or 5 to 50 nucleic acids. When PCR conditions allow for amplification of all allelic types, the types can be distinguished by hybridization with an allelic specific probe, by restriction endonuclease digestion, by electrophoresis on denaturing gradient gels, or other techniques.

Ligase chain reaction (LCR) is also carried out in accordance with known techniques. See, e.g., R. Weiss, Science 254, 1292 (1991). In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to spnK or from sequences which can be generated from such genes in accordance with standard techniques.

It will be appreciated that the detecting steps described herein may be carried out directly or indirectly. Other means of indirectly determining allelic type include measuring polymorphic markers that are linked to the particular functional polymorphism, as has been demonstrated for the VNTR (variable number tandem repeats).

Molecular biology comprises a wide variety of techniques for the analysis of nucleic acid and protein sequences. Many of these techniques and procedures form the basis of clinical diagnostic assays and tests. These techniques include nucleic acid hybridization analysis, restriction enzyme analysis, genetic sequence analysis, and the separation and purification of nucleic acids and proteins (See, e.g., J. Sambrook, E. F. Fritsch, and T. Maniatis, Molecular Cloning: A Laboratory Manual, 2 Ed., Cold spring Harbor Laboratory Press, Cold Spring Harbor, N.Y., 1989).

Most of these techniques involve carrying out numerous operations (e.g., pipetting, centrifugation, and electrophoresis) on a large number of samples. They are often complex and time consuming, and generally require a high degree of accuracy. Many techniques are limited in their application by a lack of sensitivity, specificity, or reproducibility.

Nucleic acid hybridization analysis generally involves the detection of a very small number of specific target nucleic acids (DNA or RNA) with an excess of probe DNA, among a relatively large amount of complex non-target nucleic acids. A reduction in the complexity of the nucleic acid in a sample is helpful to the detection of low copy numbers (i.e. 10,000 to 100,000) of nucleic acid targets. DNA complexity reduction is achieved to some degree by amplification of target nucleic acid sequences. (See, M. A. Innis et al., PCR Protocols: A Guide to Methods and Applications, Academic Press, 1990, Spargo et al., 1996, Molecular & Cellular Probes, in regard to SDA amplification). This is because amplification of target nucleic acids results in an enormous number of target nucleic acid sequences relative to non-target sequences thereby improving the subsequent target hybridization step.

The hybridization step involves placing the prepared DNA sample in contact with a specific reporter probe at set optimal conditions for hybridization to occur between the target DNA sequence and probe. Hybridization may be performed in any one of a number of formats. For example, multiple sample nucleic acid hybridization analysis has been conducted in a variety of filter and solid support formats (See Beltz et al., Methods in Enzymology, Vol. 100, Part et al., Eds., Academic Press, New York, Chapter 19, pp. 266-308, 1985). One format, the so-called “dot blot” hybridization, involves the non-covalent attachment of target DNAs to a filter followed by the subsequent hybridization to a radioisotope labeled probe(s). “Dot blot” hybridization gained wide-spread use over the past two decades during which time many versions were developed (see Anderson and Young, in Nucleic Acid Hybridization—A Practical Approach, Hames and Higgins, Eds., IRL Press, Washington, D.C. Chapter 4, pp. 73-111, 1985). For example, the dot blot method has been developed for multiple analyses of genomic mutations (EPA 0228075 to Nanibhushan et al.) and for the detection of overlapping clones and the construction of genomic maps (U.S. Pat. No. 5,219,726 to Evans).

Additional techniques for carrying out multiple sample nucleic acid hybridization analysis include micro-formatted multiplex or matrix devices (e.g., DNA chips) (see M. Barinaga, 253 Science, pp. 1489, 1991; W. Bains, 10 Bio/Technology, pp. 757-758, 1992). These methods usually attach specific DNA sequences to very small specific areas of a solid support, such as micro-wells of a DNA chip. These hybridization formats are micro-scale versions of the conventional “dot blot” and “sandwich” hybridization systems.

The micro-formatted hybridization can be used to carry out “sequencing by hybridization” (SBH) (see M. Barinaga, 253 Science, pp. 1489, 1991; W. Bains, 10 Bio/Technology, pp. 757-758, 1992). SBH makes use of all possible n-nucleotide oligomers (n-mers) to identify n-mers in an unknown DNA sample, which are subsequently aligned by algorithm analysis to produce the DNA sequence (See, Drmanac U.S. Pat. No. 5,202,231).

There are two formats for carrying out SBH. The first format involves creating an array of all possible n-mers on a support, which is then hybridized with the target sequence. The second format involves attaching the target sequence to a support, which is sequentially probed with all possible n-mers. Southern, (United Kingdom Patent Application GB 8810400, 1988; E. M. Southern et al., 13 Genomics 1008, 1992), proposed using the first format to analyze or sequence DNA. Southern identified a known single point mutation using PCR amplified genomic DNA. Southern also described a method for synthesizing an array of oligonucleotides on a solid support for SBH. Drmanac et al., (260 Science 1649-1652, 1993), used a second format to sequence several short (116 bp) DNA sequences. Target DNAs were attached to membrane supports (“dot blot” format). Each filter was sequentially hybridized with 272 labeled 10-mer and 1-mer oligonucleotides. Wide ranges of stringency conditions were used to achieve specific hybridization for each n-mer probe. Washing times varied from 5 minutes to overnight using temperatures from 0° C. to 16° C. Most probes required 3 hours of washing at 16° C. The filters had to be exposed from 2 to 18 hours in order to detect hybridization signals.

Generally, a variety of methods are available for detection and analysis of the hybridization events. Depending on the reporter group (fluorophore, enzyme, radioisotope, etc.) used to label the DNA probe, detection and analysis are carried out fluorimetrically, calorimetrically, or by autoradiography. By observing and measuring emitted radiation, such as fluorescent radiation or particle emission, information may be obtained about the hybridization events. Even when detection methods have very high intrinsic sensitivity, detection of hybridization events is difficult because of the background presence of non-specifically bound materials. Thus, detection of hybridization events is dependent upon how specific and sensitive hybridization can be made. Concerning genetic analysis, several methods have been developed that have attempted to increase specificity and sensitivity.

One form of genetic analysis is analysis centered on elucidation of single nucleic acid polymorphisms or (“SNPs”). Factors favoring the usage of SNPs are their high abundance in the human genome (especially compared to short tandem repeats, (STRs)), their frequent location within coding or regulatory regions of genes (which can affect protein structure or expression levels), and their stability when passed from one generation to the next (Landegren et al., Genome Research, Vol. 8, pp. 769-776, 1998).

A SNP is defined as any position in the genome that exists in two variants and the most common variant occurs less than 99% of the time. In order to use SNPs as widespread genetic markers, it is crucial to be able to genotype them easily, quickly, accurately, and cost-effectively. Numerous techniques are currently available for typing SNPs (for review, see Landegren et al., Genome Research, Vol. 8, pp. 769-776, (1998), all of which require target amplification. They include direct sequencing (Carothers et al., BioTechniques, Vol. 7, pp. 494-499, 1989), single-strand conformation polymorphism (Orita et al., Proc. Natl. Acad. Sci. USA, Vol. 86, pp. 2766-2770, 1989), allele-specific amplification (Newton et al., Nucleic Acids Research, Vol. 17, pp. 2503-2516, (1989), restriction digestion (Day and Humphries, Analytical Biochemistry, Vol. 222, pp. 389-395, 1994), and hybridization assays. In their most basic form, hybridization assays function by discriminating short oligonucleotide reporters against matched and mismatched targets. Many adaptations to the basic protocol have been developed. These include ligation chain reaction (Wu and Wallace, Gene, Vol. 76, pp. 245-254, 1989) and minisequencing (Syvanen et al., Genomics, Vol. 8, pp. 684-692, 1990). Other enhancements include the use of the 5′-nuclease activity of Taq DNA polymerase (Holland et al., Proc. Natl. Acad. Sci. USA, Vol. 88, pp. 7276-7280, 1991), molecular beacons (Tyagi and Kramer, Nature Biotechnology, Vol. 14, pp. 303-308, 1996), heat denaturation curves (Howell et al., Nature Biotechnology, Vol. 17, pp. 87-88, 1999) and DNA “chips” (Wang et al., Science, Vol. 280, pp. 1077-1082, 1998).

An additional phenomenon that can be used to distinguish SNPs is the nucleic acid interaction energies or base-stacking energies derived from the hybridization of multiple target specific probes to a single target. (See, R. Ornstein et al., “An Optimized Potential Function for the Calculation of Nucleic Acid Interaction Energies,” Biopolymers, Vol. 17, 2341-2360 (1978); J. Norberg and L. Nilsson, Biophysical Journal, Vol. 74, pp. 394-402, (1998); and J. Pieters et al., Nucleic Acids Research, Vol. 17, no. 12, pp. 4551-4565 (1989)). This base-stacking phenomenon is used in a unique format in the current invention to provide highly sensitive Tm differentials allowing the direct detection of SNPs in a nucleic acid sample.

Additional methods have been used to distinguish nucleic acid sequences in related organisms or to sequence DNA. For example, U.S. Pat. No. 5,030,557 by Hogan et al. disclosed that the secondary and tertiary structure of a single stranded target nucleic acid may be affected by binding “helper” oligonucleotides in addition to “probe” oligonucleotides causing a higher Tm to be exhibited between the probe and target nucleic acid. That application however was limited in its approach to using hybridization energies only for altering the secondary and tertiary structure of self-annealing RNA strands, which if left unaltered would tend to prevent the probe from hybridizing to the target.

With regard to DNA sequencing, K. Khrapko et al., Federation of European Biochemical Societies Letters, Vol. 256, no. 1, 2, pp. 118-122 (1989), for example, disclosed that continuous stacking hybridization resulted in duplex stabilization. Additionally, J. Kieleczawa et al., Science, Vol. 258, pp. 1787-1791 (1992), disclosed the use of contiguous strings of hexamers to prime DNA synthesis wherein the contiguous strings appeared to stabilize priming. Likewise, L. Kotler et al., Proc. Natl. Acad. Sci. USA, Vol. 90, pp. 4241-4245, (1993) disclosed sequence specificity in the priming of DNA sequencing reactions by use of hexamer and pentamer oligonucleotide modules. Further, S. Parinov et al., Nucleic Acids Research, Vol. 24, no. 15, pp. 2998-3004, (1996), disclosed the use of base-stacking oligomers for DNA sequencing in association with passive DNA sequencing microchips. Moreover, G. Yershov et al., Proc. Natl. Acad. Sci. USA, Vol. 93, pp. 4913-4918 (1996), disclosed the application of base-stacking energies in SBH on a passive microchip. In Yershov's example, 10-mer DNA probes were anchored to the surface of the microchip and hybridized to target sequences in conjunction with additional short probes, the combination of which appeared to stabilize binding of the probes. In that format, short segments of nucleic acid sequence could be elucidated for DNA sequencing. Yershov further noted that in their system the destabilizing effect of mismatches was increased using shorter probes (e.g., 5-mers). Use of such short probes in DNA sequencing provided the ability to discern the presence of mismatches along the sequence being probed rather than just a single mismatch at one specified location of the probe/target hybridization complex. Use of longer probes (e.g., 8-mer, 10-mer, and 13-mer oligos) was less functional for such purposes.

An additional example of methodologies that have used base-stacking in the analysis of nucleic acids includes U.S. Pat. No. 5,770,365 by Lane et al., wherein is disclosed a method of capturing nucleic acid targets using a unimolecular capture probe having a single stranded loop and a double stranded region which acts in conjunction with a binding target to stabilize duplex formation by stacking energies.

The nucleotide sequence may be conveniently modified by site-directed mutagenesis in accordance with conventional methods. Alternatively, the nucleotide sequence may be prepared by chemical synthesis, including but not limited to, by using an oligonucleotide synthesizer, wherein oligonucleotides are designed based on the amino acid sequence of the desired polypeptide, and preferably selecting those codons that are favored in the host cell in which the recombinant polypeptide will be produced.

Novel spinosyns can also be produced by mutagenesis of the cloned genes, and substitution of the mutated genes for their unmutated counterparts in a spinosyn-producing organism. Mutagenesis may involve, for example: 1) deletion or inactivation of a KR, DH or ER domain so that one or more of these functions is blocked and the strain produces a spinosyn having a lactone nucleus with a double bond, a hydroxyl group, or a keto group that is not present in the nucleus of spinosyn A (see Donadio et al., 1993); 2) replacement of an AT domain so that a different carboxylic acid is incorporated in the lactone nucleus (see Ruan et al., 1997); 3) addition of a KR, DH, or ER domain to an existing PKS module so that the strain produces a spinosyn having a lactone nucleus with a saturated bond, hydroxyl group, or double bond that is not present in the nucleus of spinosyn A; or 4) addition or subtraction of a complete PKS module so that the cyclic lactone nucleus has a greater or lesser number of carbon atoms. A hybrid PKS can be created by replacing the spinosyn PKS loading domain with heterologous PKS loading. See, e.g., U.S. Pat. No. 7,626,010. It has further been noted that spinosyns via modification of the sugars that are attached to the spinosyn lactone backbone can include modifications of the rhamnose and/or forosamine moiety or attachment of different deoxy sugars. The Salas group in Spain demonstrated that novel polyketide compounds can be produced by substituting the existing sugar molecule with different sugar molecules. Rodriguez et al. J. Mol. Microbiol. Biotechnol. 2000 July; 2(3):271-6. The examples that follow throughout the application help to illustrate the use of mutagenesis to produce a spinosyn with modified functionality.

The DNA from the spinosyn gene cluster region can be used as a hybridization probe to identify homologous sequences. Thus, the DNA cloned here could be used to locate additional plasmids from the Saccharopolyspora spinosa gene libraries which overlap the region described here but also contain previously uncloned DNA from adjacent regions in the genome of Saccharopolyspora spinosa. In addition, DNA from the region cloned here may be used to identify non-identical but similar sequences in other organisms. Hybridization probes are normally at least about 20 bases long and are labeled to permit detection.

Various types of mutagenesis can be used in the invention for a variety of purposes. They include, but are not limited to, site-directed, random point mutagenesis, homologous recombination, DNA shuffling or other recursive mutagenesis methods, chimeric construction, mutagenesis using uracil containing templates, oligonucleotide-directed mutagenesis, phosphorothioate-modified DNA mutagenesis, mutagenesis using gapped duplex DNA or the like, or any combination thereof. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, including but not limited to, involving chimeric constructs, are also included in the present invention. In one embodiment, mutagenesis can be guided by known information of the naturally occurring molecule or altered or mutated naturally occurring molecule, including but not limited to, sequence, sequence comparisons, physical properties, crystal structure or the like.

The texts and examples found herein describe these procedures. Additional information is found in the following publications and references cited within: Ling et al., Approaches to DNA mutagenesis: an overview, Anal Biochem. 254(2): 157-178 (1997); Dale et al., Oligonucleotide-directed random mutagenesis using the phosphorothioate method, Methods Mol. Biol. 57:369-374 (1996); Smith, In vitro mutagenesis, Ann. Rev. Genet. 19:423-462 (1985); Botstein & Shortle, Strategies and applications of in vitro mutagenesis, Science 229:1193-1201 (1985); Carter, Site-directed mutagenesis, Biochem. J. 237:1-7 (1986); Kunkel, The efficiency of oligonucleotide directed mutagenesis, in Nucleic Acids & Molecular Biology (Eckstein, F. and Lilley, D. M. J. eds., Springer Verlag, Berlin) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Bass et al., Mutant Trp repressors with new DNA-binding specificities, Science 242:240-245 (1988); Methods in Enzymol. 100: 468-500 (1983); Methods in Enzymol. 154: 329-350 (1987); Zoller & Smith, Oligonucleotide-directed mutagenesis using M13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); Zoller & Smith, Oligonucleotide-directed mutagenesis: a simple method using two oligonucleotide primers and a single-stranded DNA template, Methods in Enzymol. 154:329-350 (1987); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Kramer et al., The gapped duplex DNA approach to oligonucleotide-directed mutation construction, Nucl. Acids Res. 12: 9441-9456 (1984); Kramer & Fritz Oligonucleotide-directed construction of mutations via gapped duplex DNA, Methods in Enzymol. 154:350-367 (1987); Kramer et al., Improved enzymatic in vitro reactions in the gapped duplex DNA approach to oligonucleotide-directed construction of mutations, Nucl. Acids Res. 16: 7207 (1988); Fritz et al., Oligonucleotide-directed construction of mutations: a gapped duplex DNA procedure without enzymatic reactions in vitro, Nucl. Acids Res. 16: 6987-6999 (1988); Kramer et al., Point Mismatch Repair, Cell 38:879-887 (1984); Carter et al., Improved oligonucleotide site-directed mutagenesis using M13 vectors, Nucl. Acids Res. 13: 4431-4443 (1985); Carter, Improved oligonucleotide-directed mutagenesis using M13 vectors, Methods in Enzymol. 154: 382-403 (1987); Eghtedarzadeh & Henikoff, Use of oligonucleotides to generate large deletions, Nucl. Acids Res. 14: 5115 (1986); Wells et al., Importance of hydrogen-bond formation in stabilizing the transition state of subtilisin, Phil. Trans. R. Soc. Lond. A 317: 415-423 (1986); Nambiar et al., Total synthesis and cloning of a gene coding for the ribonuclease S protein, Science 223: 1299-1301 (1984); Sakamar and Khorana, Total synthesis and expression of a gene for the a-subunit of bovine rod outer segment guanine nucleotide-binding protein (transducin), Nucl. Acids Res. 14: 6361-6372 (1988); Wells et al., Cassette mutagenesis: an efficient method for generation of multiple mutations at defined sites, Gene 34:315-323 (1985); Grundstrom et al., Oligonucleotide-directed mutagenesis by microscale ‘shot-gun’ gene synthesis, Nucl. Acids Res. 13: 3305-3316 (1985); Mandecki, Oligonucleotide-directed double-strand break repair in plasmids of Escherichia coli: a method for site-specific mutagenesis, Proc. Natl. Acad. Sci. USA, 83:7177-7181 (1986); Arnold, Protein engineering for unusual environments, Current Opinion in Biotechnology 4:450-455 (1993); Sieber, et al., Nature Biotechnology, 19:456-460 (2001). W. P. C. Stemmer, Nature 370, 389-91 (1994); and, I. A. Lorimer, I. Pastan, Nucleic Acids Res. 23, 3067-8 (1995). Additional details on many of the above methods can be found in Methods in Enzymology Volume 154, which also describes useful controls for trouble-shooting problems with various mutagenesis methods.

The terms “homology” or “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available on the internet at the accelrys website, more specifically at http://www.accelrys.com), using either a Blossom 62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available on the internet at the accelrys website, more specifically at http://www.accelrys.com), using a NWSgapdna.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (CABIOS, 4: 11-17 (1989) which has been incorporated into the ALIGN program (version 2.0) (available on the internet at the vega website, more specifically ALIGN-IGH Montpellier, or more specifically at http://vega.igh.cnrs.fr/bin/align-guess.cgi) using a PAM120 weight residue table, a gap length penalty of 12 and a gap penalty of 4.

The nucleic acid and protein sequences of the present invention may further be used as a “query sequence” to perform a search against public databases to, for example, identify other family members or related sequences. Such searches may be performed using the BLASTN and BLASTX programs (version 2.0) of Altschul, et al. (1990) J. Mol. Biol. 215:403-10. BLAST nucleotide searches may be performed with the BLASTN program, score=100, word length=12 to obtain nucleotide sequences homologous to the nucleic acid molecules of the present invention. BLAST protein searches may be performed with the BLASTX program, score=50, word length=3 to obtain amino acid sequences homologous to the protein molecules of the present invention. To obtain gapped alignments for comparison purposes, Gapped BLAST may be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) may be used. (Available on the internet at the ncbi website, more specifically at http://www.ncbi.nlm.nih.gov).

Other embodiments of the present invention can include manipulations in the spnK gene that may include single or multiple nucleotide base deletion(s) which can disrupt the normal reading frame of spnK. Such deletions may include anywhere from 1 to 1194 nucleotides. Such deletion affects the normal reading frame of spnK resulting in the production of a spinetoram precursor producing strain.

Another embodiment of the present invention can include manipulations in the spnK gene that can include single or multiple nucleotide insertion(s) within the spnK coding region which disrupts the normal reading frame of spnK. Such insertion affects the normal reading frame of spnK resulting in the production of a spinetoram precursor producing strain.

Additional embodiments of the present invention can include manipulations in the spnK gene that include the use of antisense or sense technology to abolish or significantly interfere with the production of the SpnK protein. The person skilled in the art knows how to achieve an antisense and a cosuppression effect. For example, the method of cosuppression inhibition has been described in Jorgensen (Trends Biotechnol. 8 (1990), 340-344), Niebel et al., (Curr. Top. Microbiol. Immunol. 197 (1995), 91-103), Flavell et al. (Curr. Top. Microbiol. Immunol. 197 (1995), 43-46), Palaqui and Vaucheret (Plant. Mol. Biol. 29 (1995), 149-159), Vaucheret et al., (Mol. Gen. Genet. 248 (1995), 311-317), de Borne et al. (Mol. Gen. Genet. 243 (1994), 613-621).

The present invention therefore further provides methods of gene silencing, by expressing in an organism, such as S. spinosa, a nucleic acid having an inverted repeat 5′ or 3′ to a sense or antisense targeting sequence, wherein the sense or antisense targeting sequence has substantial sequence identity to the target gene to be suppressed, but the inverted repeat is not related by sequence to the target gene. In another embodiment, the heterologous inverted repeat is flanked by a 5′ and 3′ targeting sequence.

The gene silencing construct can be expressed in the organism of choice, e.g., a bacterial cell, a fungal cell, a eukaryotic cell, e.g., a plant cell or a mammalian cell.

Suitable expression vectors for use in the present invention include prokaryotic and eukaryotic vectors (e.g., plasmid, phagemid, or bacteriophage), include mammalian vectors and plant vectors. Suitable prokaryotic vectors include plasmids such as, but not limited to, those commonly used for DNA manipulation in Actinomyces, (for example pSET152, pOJ260, pIJ101, pJV1, pSG5, pHJL302, pSAM2, pKC1250. Such plasmids are disclosed by Kieser et al. (“Practical Streptomyces Genetics,” 2000). Other suitable vectors can include plasmids such as those capable of replication in E. coli (for example, pBR322, ColE1, pSC101, PACYC 184, itVX, pRSET, pBAD (Invitrogen, Carlsbad, Calif.) and the like). Such plasmids are disclosed by Sambrook (cf. “Molecular Cloning: A Laboratory Manual,” second edition, edited by Sambrook, Fritsch, & Maniatis, Cold Spring Harbor Laboratory, (1989)) and many such vectors are commercially available. Bacillus plasmids include pC194, pC221, pT127, and the like, and are disclosed by Gryczan (In: The Molecular Biology of the Bacilli, Academic Press, NY (1982), pp. 307-329). Suitable Streptomyces plasmids include pli101 (Kendall et al., J. Bacteriol. 169:4177-4183, 1987), and Streptomyces bacteriophages include but not limited to such as ψC31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704, 1986), and Izaki (Jpn. J. Bacteriol. 33:729-742, 1978).

Suppression of the expression of particular genes is an important tool both for research and for the development of genetically engineered organisms more fitted for a particular purpose. Gene silencing can be accomplished by the introduction of a transgene corresponding to the gene of interest in the antisense orientation relative to its promoter (see, e.g., Sheehy et al., Proc. Nat'l Acad. Sci. USA 85:8805 8808 (1988); Smith et al., Nature 334:724 726 (1988)), or in the sense orientation relative to its promoter (Napoli et al., Plant Cell 2:279 289 (1990); van der Krol et al., Plant Cell 2:291 299 (1990); U.S. Pat. No. 5,034,323; U.S. Pat. No. 5,231,020; and U.S. Pat. No. 5,283,184), both of which lead to reduced expression of the transgene as well as the endogenous gene.

Posttranscriptional gene silencing has been reported to be accompanied by the accumulation of small (20 to 25 nucleotide) fragments of antisense RNA, which can be synthesized from an RNA template and represent the specificity and mobility determinants of the process (Hamilton & Baulcombe, Science 286:950 952 (1999)). It has become clear that in a range of organisms the introduction of dsRNA (double-stranded RNA) is an important component leading to gene silencing (Fire et al., Nature 391:806 811 (1998); Timmons & Fire, Nature 395:854 (1998); WO99/32619; Kennerdell & Carthew, Cell 95:1017 1026 (1998); Ngo et al., Proc. Nat'l Acad. Sci. USA 95:14687 14692 (1998); Waterhouse et al., Proc. Nat'l Acad. Sci. USA 95:13959 13964 (1998); WO99/53050; Cogoni & Macino, Nature 399:166 169 (1999); Lohmann et al., Dev. Biol. 214:211 214 (1999); Sanchez-Alvarado & Newmark, Proc. Nat'l Acad. Sci. USA 96:5049 5054 (1999)). In bacteria the suppressed gene does not need to be an endogenous bacterial gene, since both reporter transgenes and virus genes are subject to posttranscriptional gene silencing by introduced transgenes (English et al., Plant Cell 8:179 188 (1996); Waterhouse et al, supra). However, in all of the above cases, some sequence similarity may be preferred between the introduced transgene and the gene that is suppressed.

In prior examples, introduction of a sense transgene consisting of the 5′-UTR (“untranslated region”), coding region and 3′-UTR of an ACC oxidase gene under the control of the CaMV ³⁵S promoter resulted in reduced ACC oxidase enzyme activity in 15% of a population of tomato plants (Hamilton et al., Plant J. 15:737 746 (1998); WO98/53083). However, if inverted and sense repeats of part of the 5′-UTR of this ACC oxidase were included in the construct, suppression was observed in 96% of the plants (Hamilton et al., supra). In addition, suppression of another ACC oxidase gene related in sequence to the coding region of the transgene but not to the 5′-UTR of the transgene was suppressed, showing that double-stranded RNA of any part of the transcript targets the entire RNA transcript for degradation. In addition, high frequency and high level posttranscriptional gene silencing have been found by introduction either of constructs containing inverted repeats of the coding regions of virus or reporter genes, or by crossing together plants expressing the sense and antisense transcripts of the coding region of the target gene (Waterhouse et al., Proc. Nat'l Acad. Sci. USA 95:13959 13964 (1998)). Similar results were obtained by expression of sense and antisense transgenes under the control of different promoters in the same plant (Chuang & Meyerowitz, Proc. Nat'l Acad. Sci. USA 97:4985 4990 (2000)).

Other embodiments of the present invention can include manipulations in the spnK gene that include gene silencing. The phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi), a defined, though partially characterized pathway whereby small inhibitory RNA (siRNA) act in concert with host proteins (e.g., the RNA induced silencing complex, RISC) to degrade messenger RNA (mRNA) in a sequence-dependent fashion. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

Additional embodiments include single or multiple amino acid substitution(s) in the active site or the substrate binding site of the spnK gene that disable the spnK gene and result in spinosyn J/L production. In general, those skilled in the art will appreciate that minor deletions or substitutions may be made to the amino acid sequences of peptides of the present invention without unduly adversely affecting the activity thereof. Thus, proteins and peptides containing such deletions or substitutions are a further aspect of the present invention. In peptides containing substitutions or replacements of amino acids, one or more amino acids of a peptide sequence may be replaced by one or more other amino acids wherein such replacement does not affect the function of that sequence. Such changes can be guided by known similarities between amino acids in physical features such as charge density, hydrophobicity/hydrophilicity, size and configuration, so that amino acids are substituted with other amino acids having essentially the same functional properties. For example: Ala may be replaced with Val or Ser; Val may be replaced with Ala, Leu, Met, or Ile, preferably Ala or Leu; Leu may be replaced with Ala, Val or Ile, preferably Val or lie; Gly may be replaced with Pro or Cys, preferably Pro; Pro may be replaced with Gly, Cys, Ser, or Met, preferably Gly, Cys, or Ser; Cys may be replaced with Gly, Pro, Ser, or Met, preferably Pro or Met; Met may be replaced with Pro or Cys, preferably Cys; His may be replaced with Phe or Gln, preferably Phe; Phe may be replaced with His, Tyr, or Trp, preferably His or Tyr; Tyr may be replaced with His, Phe or Trp, preferably Phe or Trp; Trp may be replaced with Phe or Tyr, preferably Tyr; Asn may be replaced with Gln or Ser, preferably Gln; KGln may be replaced with His, Lys, Glu, Asn, or Ser, preferably Asn or Ser; Ser may be replaced with Gln, Thr, Pro, Cys or Ala; Thr may be replaced with Gln or Ser, preferably Ser; Lys may be replaced with Gln or Arg; Arg may be replaced with Lys, Asp or Glu, preferably Lys or Asp; Asp may be replaced with Lys, Arg, or Glu, preferably Arg or Glu; and Glu may be replaced with Arg or Asp, preferably Asp. Once made, changes can be routinely screened to determine their effects on function.

Other embodiments of the present invention can include manipulations in the spnK gene that include the manipulation of ribosome binding sites (RBS). The ribosome binding site (labeled as Shine-Dalgarno), which is located upstream of the spnK coding sequence, can be manipulated so that the spnK gene is disrupted resulting in the production of a spinetoram precursor producing strain.

Additional embodiments of the present invention can include enzymatic inhibition affecting multiple signaling pathways for the spnK gene that can result in the production of a spinetoram producing strain. Methods of detecting enzymatic activity associated with a target can include the use enzyme-linked assays.

Another embodiment of the present invention includes interruption of the promoter sequence that encodes for the spnK gene. Such interruption can be through any type of manipulation including but not limited to truncations, deletions, point mutations, and insertions. Such manipulations may be in or out of frame. Such manipulations result in producing a spinetoram producing strain.

The present invention is explained in greater detail in the following non-limiting examples.

### Example 1

### Generation of Point Mutations within spnK

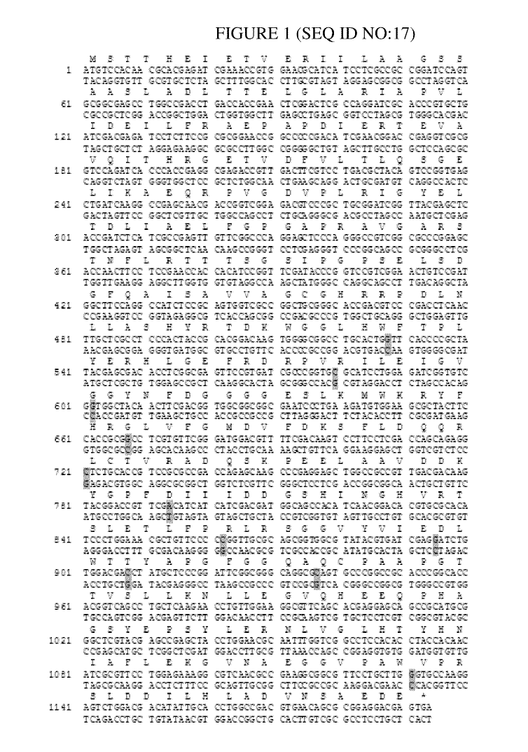

Point mutations within the spnK gene were generated via random mutagenesis of a Saccharopolyspora spinosa A and D producing strain (Kieser et al., 2000). Mutant strains producing spinosyn J and L instead of spinosyn A and D were further characterized via PCR amplification of the spnK gene followed by DNA sequencing. The spnK gene was PCR amplified with spnKF (SEQ ID NO: 1; GGGAATTCCATATGTCCACAACGCACGAGATCGA) and spnKR (SEQ ID NO: 2; GCCGCTCGAGCTCGTCCTCCGCGCTGTTCACGTCS) using the FailSafe PCR System (Epicentre Biotechnologies; Madison, Wis.). The resulting PCR product was purified using MoBio Ultraclean PCR Clean-up DNA Purification Kit (MoBio Laboratories; Solana Beach, Calif.) and was cloned into the TA cloning vector using T4 DNA ligase (Invitrogen Life Technologies; Carlsbad, Calif.). Bacterial colonies which putatively contained the PCR product were isolated and confirmed via restriction enzyme digestion. DNA sequencing of the positive plasmid clones was performed as described by manufacturer's protocol using the CEQ™ DTCS-Quick Start Kit (Beckman-Coulter; Palo Alto, Calif.). Reactions were purified using Performa DTR Gel Filtration Cartridges (Edge BioSystems; Gaithersburg, Md.) as described by manufacturer protocols. Sequence reactions were analyzed on a Beckman-Coulter CEQ™ 2000 XL DNA Analysis System and nucleotide characterization performed using SEQUENCHER™ (Gene Codes Corporation; Ann Arbor, Mich.). The sequencing results confirmed the location of point mutations within the spnK gene sequence. The resulting point mutations are listed in Table 1 and are shaded in FIG. 1.

Fermentation of the spnK mutant strains of Saccharopolyspora spinosa can be performed under conditions described by Burns et al., (WO 2003070908). Analysis of the fermentation broth for the presence of spinosyn factors can be carried out under conditions described by Baltz et al., (U.S. Pat. No. 6,143,526). To confirm the presence of the spinosyn factors in the supernatant, extracts of the fermentation broth were dried down in a SpeedVac overnight followed by partition of the residue between water and ether. The ether layer was dried by evaporation under N₂ stream. The sample was then dissolved in acetone-d₆ and transferred to an NMR tube for 1D proton NMR acquisition. The NMR profiles were compared to those of spinosyn standards. The NMR results indicated a presence of an excess of J/L over A/D. Fermentation of the strains which contain the point mutation produced a spinosyn mixture containing spinosyn J and L, as compared to the control Saccharopolyspora spinosa which produced a spinosyn mixture containing spinosyn A and D.

TABLE 1
List of point mutations, their location within spnK and the spinsoyn
compounds produced by these strains during fermentation.

| Strain | | Location of | Spinosyn Compound |
|---|---|---|---|
| # | Resulting Mutation | Mutation | Production |
| 1. | TGG (W) → TGA | Base pair 528 | Spinosyn J and L |
| | stop codon | | |
| 2. | CGC (R) → TGC (C) | Base pair 589 | Spinosyn J and L |
| 3. | GGT (G) → GAT (D) | Base pair 602 | Spinosyn J and L |
| 4. | GGC (G) → GAC (D) | Base pair 668 | Spinosyn J and L |
| 5. | CTC (L) → TTC (F) | Base pair 721 | Spinosyn J and L |
| 6. | GAC (D) → GGC (G) | Base pair 794 | Spinosyn J and L |
| 7. | CGG (R) → TGG (W) | Base pair 862 | Spinosyn J and L |
| 8. | GAT (D) → AAT (N) | Base pair 895 | Spinosyn J and L |
| 9. | ACC (T) → ATC (I) | Base pair 908 | Spinosyn J and L |
| 10. | CAG (Q) → TAG | Base pair 937 | Spinosyn J and L |
| | stop codon | | |
| 11. | TGG (W) → TGA | Base pair 1131 | Spinosyn J and L |
| | stop codon | | |
| Control | Wild Type Control | Not Applicable | Spinosyn A and D |

### Example 2

### Generation of a spnK Deletion Mutation

Construction of the spnK in Frame Deletion Vector

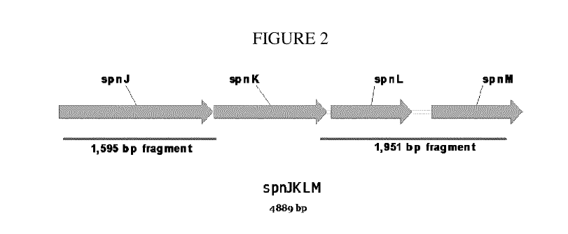

A 1,595 bp DNA fragment was PCR amplified using the genomic DNA of a spinosyn A and D producing strain (Hopwood et al., 1985). This fragment spanned the start codon of spnK and contained the spnJ coding region without the 5′ end of spnJ (FIG. 2). The PCR reaction was completed using the FailSafe PCR kit (Epicentre Biotechnologies; Madison, Wis.) and Forward Primer #1 (SEQ ID NO: 3; CGGTGCCCGAATTCCATGACCCG) and Reverse Primer #1 (SEQ ID NO: 4; GTGCGTTCTAGACATATGAGCTCCTCATGGCTG).

A second PCR reaction was completed which produced a 1,951 bp DNA fragment; this fragment contained the 3′ end of spnK, an intact spnL and the 5′ end of spnM (FIG. 2). The PCR reaction was completed using the FailSafe PCR kit and Forward Primer #2 (SEQ ID NO: 5; GTGCCATCTAGACTGGACGACATATTGCACCTG) and Reverse Primer #2 (SEQ ID NO: 6; GAATGCGAAGCT TACGATCTCGTCGTCCGTG). PCR products were purified using the QIAquick PCR Purification Kit (Qiagen; Valencia, Calif.) according to manufacturer's instructions.

The 1,595 bp PCR fragment was digested with EcoRI and XbaI. The 1,951 bp PCR fragment was digested with XbaI and HindIII. Upon completion of the restriction enzyme digestion the fragments were purified using the QIAquick PCR Purification Kit. The digested fragments were ligated to corresponding EcoRI and HindIII restriction sites of plasmid pOJ260 using the FastLink DNA Ligation Kit (Epicentre; Madison, Wis.) and transformed into E. coli TOP10 competent cells (Invitrogen; Carlsbad, Calif.). Colonies were selected and screened for the desired ligation product via restriction enzyme digestion and DNA sequence analysis. Positive clones were identified and a selected clone was used for subsequent in-frame deletion of spnK in Saccharopolyspora spinosa. The resulting sequence of the deleted spnK gene fragment within plasmid pOJ260 is presented in Table 2.

TABLE 2
Nucleotide sequence alignment of the deleted spnK gene.

| | | |
|---|---|---|
| spnK (SEQ ID NO: 17) | (1) | 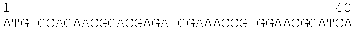 |
| spnK delete (SEQ ID NO: 9) | (1) | 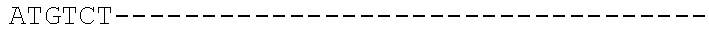 |
| | | 41 80 |
| spnK (SEQ ID NO: 17) | (41) | TCCTCGCCGCCGGATCCAGTGCGGCGAGCCTGGCCGACCT |
| spnK delete (SEQ ID NO: 9) | (7) | ---------------------------------------- |
| | | 81 120 |
| spnK (SEQ ID NO: 17) | (81) | GACCACCGAACTCGGACTCGCCAGGATCGCACCCGTGCTG |
| spnK delete (SEQ ID NO: 9) | (7) | ---------------------------------------- |
| | | 121 160 |
| spnK (SEQ ID NO: 17) | (121) | ATCGACGAGATCCTCTTCCGCGCGGAACCGGCCCCCGACA |
| spnK delete (SEQ ID NO: 9) | (7) | ---------------------------------------- |
| | | 161 200 |
| spnK (SEQ ID NO: 17) | (161) | TCGAACGGACCGAGGTCGCGGTCCAGATCACCCACCGAGG |
| spnK delete (SEQ ID NO: 9) | (7) | ---------------------------------------- |
| | | 201 240 |
| spnK (SEQ ID NO: 17) | (201) | CGAGACCGTTGACTTCGTCCTGACGCTACAGTCCGGTGAG |
| spnK delete (SEQ ID NO: 9) | (7) | ---------------------------------------- |
| | | 241 280 |
| spnK (SEQ ID NO: 17) | (241) | CTGATCAAGGCCGAGCAACGACCGGTCGGAGACGTCCCGC |
| spnK delete (SEQ ID NO: 9) | (7) | ---------------------------------------- |
| | | 281 320 |
| spnK (SEQ ID NO: 17) | (281) | TGCGGATCGGTTACGAGCTCACCGATCTCATCGCCGAGTT |
| spnK delete (SEQ ID NO: 9) | (7) | ---------------------------------------- |
| | | 321 360 |
| spnK (SEQ ID NO: 17) | (321) | GTTCGGCCCAGGAGCTCCCAGGGCCGTCGGCGCCCGGAGC |
| spnK delete (SEQ ID NO: 9) | (7) | ---------------------------------------- |
| | | 361 400 |
| spnK (SEQ ID NO: 17) | (361) | ACCAACTTCCTCCGAACCACCACATCCGGTTCGATACCCG |
| spnK delete (SEQ ID NO: 9) | (7) | ---------------------------------------- |
| | | 401 440 |
| spnK (SEQ ID NO: 17) | (401) | GTCCGTCGGAACTGTCCGATGGCTTCCAGGCCATCTCCGC |
| spnK delete (SEQ ID NO: 9) | (7) | ---------------------------------------- |
| | | 441 480 |
| spnK (SEQ ID NO: 17) | (441) | AGTGGTCGCCGGCTGCGGGCACCGACGTCCCGACCTCAAC |
| spnK delete (SEQ ID NO: 9) | (7) | ---------------------------------------- |
| | | 481 520 |
| spnK (SEQ ID NO: 17) | (481) | TTGCTCGCCTCCCACTACCGCACGGACAAGTGGGGCGGCC |
| spnK delete (SEQ ID NO: 9) | (7) | ---------------------------------------- |
| | | 521 560 |
| spnK (SEQ ID NO: 17) | (521) | TGCACTGGTTCACCCCGCTATACGAGCGACACCTCGGCGA |
| spnK delete (SEQ ID NO: 9) | (7) | ---------------------------------------- |
| | | 561 600 |
| spnK (SEQ ID NO: 17) | (561) | GTTCCGTGATCGCCCGGTGCGCATCCTGGAGATCGGTGTC |
| spnK delete (SEQ ID NO: 9) | (7) | ---------------------------------------- |
| | | 601 640 |
| spnK (SEQ ID NO: 17) | (601) | GGTGGCTACAACTTCGACGGTGGCGGCGGCGAATCCCTGA |
| spnK delete (SEQ ID NO: 9) | (7) | ---------------------------------------- |
| | | 641 680 |
| spnK (SEQ ID NO: 17) | (641) | AGATGTGGAAGCGCTACTTCCACCGCGGCCTCGTGTTCGG |
| spnK delete (SEQ ID NO: 9) | (7) | ---------------------------------------- |
| | | 681 720 |
| spnK (SEQ ID NO: 17) | (681) | GATGGACGTTTTCGACAAGTCCTTCCTCGACCAGCAGAGG |
| spnK delete (SEQ ID NO: 9) | (7) | ---------------------------------------- |
| | | 721 760 |
| spnK (SEQ ID NO: 17) | (721) | CTCTGCACCGTCCGCGCCGACCAGAGCAAGCCCGAGGAGC |
| spnK delete (SEQ ID NO: 9) | (7) | ---------------------------------------- |
| | | 761 800 |
| spnK (SEQ ID NO: 17) | (761) | TGGCCGCCGTTGACGACAAGTACGGACCGTTCGACATCAT |
| spnK delete (SEQ ID NO: 9) | (7) | ---------------------------------------- |
| | | 801 840 |
| spnK(SEQ ID NO: 17) | (801) | CATCGACGATGGCAGCCACATCAACGGACACGTGCGCACA |
| spnK delete(SEQ ID NO: 9) | (7) | ---------------------------------------- |
| | | 841 880 |
| spnK(SEQ ID NO: 17) | (841) | TCCCTGGAAACGCTGTTCCCCCGGTTGCGCAGCGGTGGCG |
| spnK delete(SEQ ID NO: 9) | (7) | ---------------------------------------- |
| | | 881 920 |
| spnK (SEQ ID NO: 17) | (881) | TATACGTGATCGAGGATCTGTGGACGACCTATGCTCCCGG |
| spnK delete (SEQ ID NO: 9) | (7) | ---------------------------------------- |
| | | 921 960 |
| spnK (SEQ ID NO: 17) | (921) | ATTCGGCGGGCAGGCGCAGTGCCCGGCCGCACCCGGCACC |
| spnK delete (SEQ ID NO: 9) | (7) | ---------------------------------------- |
| | | 961 1000 |
| spnK (SEQ ID NO: 17) | (961) | ACGGTCAGCCTGCTCAAGAACCTGTTGGAAGGCGTTCAGC |
| spnK delete (SEQ ID NO: 9) | (7) | ---------------------------------------- |
| | | 1001 1040 |
| spnK (SEQ ID NO: 17) | (1001) | ACGAGGAGCAGCCGCATGCGGGCTCGTACGAGCCGAGCTA |
| spnK delete (SEQ ID NO: 9) | (7) | ---------------------------------------- |
| | | 1041 1080 |
| spnK (SEQ ID NO: 17) | (1041) | CCTGGAACGCAATTTGGTCGGCCTCCACACCTACCACAAC |
| spnK delete (SEQ ID N0: 9) | (7) | ---------------------------------------- |
| | | 1081 1120 |
| spnK (SEQ ID NO: 17) | (1081) | ATCGCGTTCCTGGAGAAAGGCGTCAACGCCGAAGGCGGCG |
| spnK delete (SEQ ID NO: 9) | (7) | ---------------------------------------- |
| spnK (SEQ ID NO: 17) | (1121) | 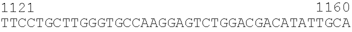 |
| spnK delete (SEQ ID NO: 9) | (7) | 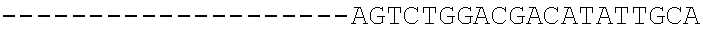 |
| spnK (SEQ ID NO: 17) | (1161) | 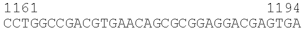 |
| spnK delete (SEQ ID NO: 9) | (27) | 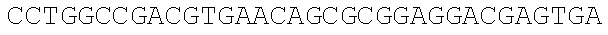 |

Therefore one spnK deletion would include the sequence:

(SEQ ID NO: 9)
ATGTCTAGACTGGACGACATATTGCACCTGGCCGACGTGAACAGCGCGG
AGGACGAGTGA.

Conjugation of spnK Deletion Vector into Saccharopolyspora spinosa

The spnK in-frame deletion construct was transformed into the E. coli conjugation donor strain ET12567/pUZ8002. A positively transformed strain was identified and used to inoculate a flask of Luria Broth media (containing appropriate antibiotics) for overnight growth at 37° C. with shaking at 225 rpm. Confirmation of plasmid identity was performed by isolating plasmid DNA and completing a restriction enzyme digestion from the E. coli donor strain. Upon confirmation that the fidelity of this clone was correct, the remaining culture was stored in 20% glycerol at −80° C. for further use.

Conjugation of the E. coli cells carrying the spnK in-frame deletion construct with Saccharopolyspora spinosa was performed according to the method described in Matsushima et al., (1994). Putative transconjugants resistant to apramycin, due to the presence of the apramycin resistance gene marker on the vector backbone of the spnK in-frame deletion construct, were selected.

Confirmation of Transconjugants and Amplification of the spnK Region to Determine the Site of Integration

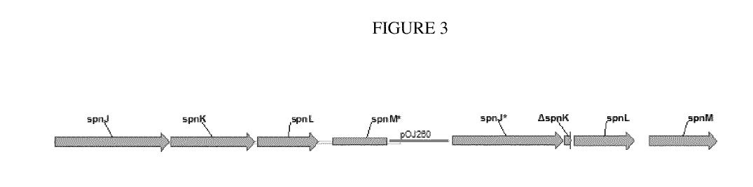

A single primary transconjugant grown on R6 media and was transferred onto Brain Heart Infusion (BHI) agar plates supplemented with 50 μg/mL of apramycin and 25 μg/mL of nalidixic acid to confirm the resistance phenotype. Mycelia of the transconjugants were inoculated from the BHI plate into Tryptic Soy Broth (TSB) media supplemented with 50 μg/mL of apramycin. The culture was incubated at 29° C. with shaking at 250 rpm for 72 hours. Mycelia were harvested after 72 hours of incubation and genomic DNA was isolated using Edge BioSystem's Genomic DNA Isolation Kit according to manufacturer's instructions (Edge Biosystems; Gaithersburgh, Md.). PCR was performed using the genomic DNA isolated from the transconjugant as template with the SpnK Del Validation 1Forward (SEQ ID NO: 7; GTTCACGGTGATTCCGGTGACTCG) and SpnK Del Validation 1Reverse (SEQ ID NO: 8; ACCTGCACTGCTTCCTGGAGCTTC) primers. In addition, genomic DNA isolated from the Saccharopolyspora spinosa parent control strain and the plasmid DNA of the spnK in-frame deletion construct were used as templates for a control PCR reaction. The PCR amplification results were sequenced. The sequencing data indicated that the spnK in-frame deletion construct integrated into the spnLM region via single crossover homologous recombination (FIG. 3). Integration at the spnLM region generated, within the chromosome, an intact copy of spnJ, spnK, spnL, and a truncated spnM upstream of the vector backbone pOJ260 and a truncated spnJ, and an intact spnL and spnM downstream of the vector backbone.

Isolation of Double Crossover spnK In-Frame Deletion Mutant

The single crossover mutant resistant to apramycin was inoculated on BHI agar plates in the absence of apramycin and incubated at 29° C. for 14 days. Spores were harvested from the plates according to Hopwood et al., (1985) and stored in 20% glycerol at −80° C. Spores were inoculated onto ten new BHI agar plates without apramycin and plates were incubated at 29° C. for 14 days. This step was repeated three times. The spore preparation was diluted to 10⁻⁶ using 20% glycerol and the diluted spores were plated on ten BHI agar plates. Plates were incubated at 29° C. for 10 days for single colony development. Individual colonies were patched onto new BHI agar plates with and without apramycin. All plates were incubated at 29° C. for 10 days for mycelial development. Colonies that did not grow on BHI agar plates containing 50 μg/mL of apramycin were identified as candidates of double crossover mutants and were selected for validation using PCR.

### Identification and Validation of Double Crossover Mutants

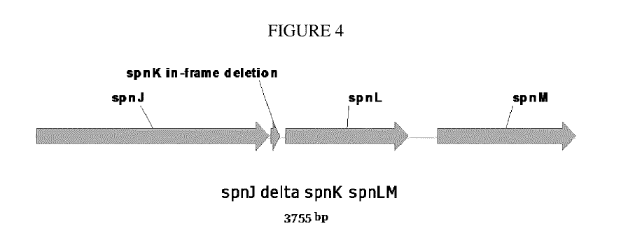

Double crossover mutants were confirmed via PCR. Primers, SpnK Del Validation 1Forward (SEQ ID NO: 7) and SpnK Del Validation 1Reverse (SEQ ID NO: 8), were designed to bind within the spnL and spnJ genes are used for PCR amplification using the FailSafe PCR System. The sizes of the PCR products were determined via agarose gel electrophoresis. Double crossover mutants which resulted in a deletion of the spnK gene were identified (FIG. 4) and selected based on the size of the PCR product. The size and DNA sequence of the PCR fragment indicates in-frame deletion of the spnK gene.

### Spinosyn Production of Double Crossover Mutants Via Shake Flask Fermentation

Fermentation of the double crossover mutant can be performed under conditions described by Burns et al., (WO 2003070908). Analysis of the fermentation broth for the presence of spinosyn factors can be carried out under conditions described by Baltz et al., (U.S. Pat. No. 6,143,526). To confirm the presence of the spinosyn factors in the supernatant, extracts of the fermentation broth were dried down in a SpeedVac overnight followed by partition of the residue between water and ether. The ether layer was dried by evaporation under N₂ stream. The sample was then dissolved in acetone-d₆ and transferred to an NMR tube for 1D proton NMR acquisition. The NMR profiles were compared to those of spinosyn standards. Fermentation of the double crossover mutant produces spinosyn J and L. The NMR results indicated a presence of an excess of J/L over A/D.

### Example 3

### Generation of a spnK Insertion Mutation

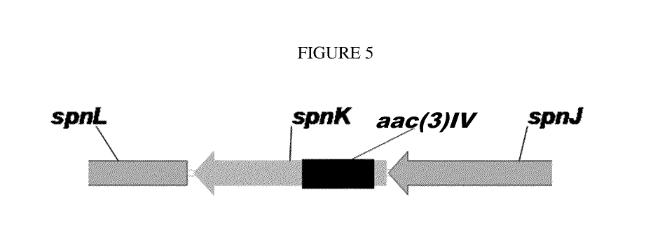

Saccharopolyspora spinosa mutants are generated via insertional mutation within the spnK gene. A DNA fragment containing an in-frame apramycin resistance gene cassette (aac(3)IV) within the spnK gene and the uninterrupted upstream and downstream spnJ and spnL gene flanking sequences is constructed (FIG. 5). The aac(3)IV gene fragment is cloned into a plasmid and transformed into the E. coli conjugation donor strain ET12567/pUZ8002. A positively transformed strain is identified and used to inoculate a flask of Luria Broth media (containing appropriate antibiotics) for overnight growth at 37° C. with shaking at 225 rpm. Confirmation of plasmid identity is performed by isolating plasmid DNA and completing a restriction enzyme digestion. Upon confirmation that the plasmid containing the apramycin insertion cassette is correct, the remaining culture is stored in 20% glycerol at −80° C.

Conjugation of the E. coli donor cells with Saccharopolyspora spinosa is performed according to the method described in Matsushima et al., (1994). The transfer of the apramycin gene cassette from E. coli and the subsequent integration of this plasmid into the genome of Saccharopolyspora spinosa is selected using resistance to apramycin.

A single primary transconjugant is grown on R6 media and transferred onto Brain Heart Infusion (BHI) agar plates supplemented with 50 μg/mL of apramycin and 25 μg/mL of nalidixic acid to confirm the resistance phenotype. Mycelia of the transconjugants are inoculated from the BHI plate into Tryptic Soy Broth (TSB) media supplemented with 50 μg/mL of apramycin. The culture is incubated at 29° C. with shaking at 250 rpm for 72 hours. Mycelia are harvested after 72 hours of incubation and genomic DNA is isolated using Edge BioSystem's Genomic DNA Isolation Kit according to manufacturer's instructions (Edge Biosystems; Gaithersburgh, Md.). PCR is performed using the genomic DNA isolated from the transconjugant as template. The desired PCR product is cloned into a plasmid using the TOPO® Cloning Technology (Invitrogen; Carlsbad Calif.). Bacterial colonies which putatively contain the PCR product, cloned within the TOPO® vector, are isolated and confirmed via restriction enzyme digestion. DNA sequencing of the positive plasmid clones is performed. The sequencing results indicate that the apramycin insertion cassette is integrated within the spnK gene of Saccharopolyspora spinosa via double crossover homologous recombination. The resulting insertion via homologous recombination disrupts the transcription of spnK thereby abolishing the spnK gene function.

Fermentation of the spnK mutant strains of Saccharopolyspora spinosa can be performed under conditions described by Burns et al., (WO 2003070908). Analysis of the fermentation broth for the presence of spinosyn factors can be carried out under conditions described by Baltz et al., (U.S. Pat. No. 6,143,526). To confirm the presence of the spinosyn factors in the supernatant, extracts of the fermentation broth are dried down in a SpeedVac overnight followed by partition of the residue between water and ether. The ether layer is dried by evaporation under N₂ stream. The sample is then dissolved in acetone-d₆ and transferred to an NMR tube for 1D proton NMR acquisition. The NMR profiles are compared to those of spinosyn standards. The NMR results indicated a presence of an excess of J/L over A/D. Fermentation of the strains which contain the insertion mutation produces a spinosyn mixture containing spinosyn J and L, as compared to the control Saccharopolyspora spinosa which produces a spinosyn mixture containing spinosyn A and D.

### Example 4

### Disruption of the spnK Shine-Dalgarno Sequence

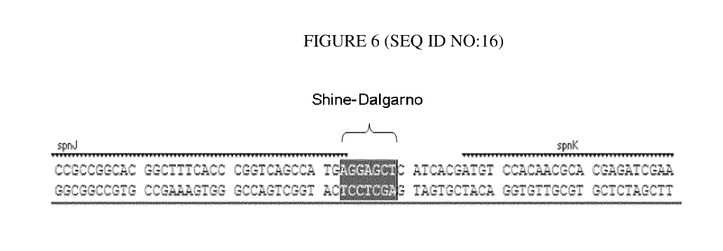

The Shine-Dalgarno sequence located upstream of spnK (FIG. 6) is disrupted thereby resulting in reduced translation of the spnK mRNA. A mutant strain of Saccharopolyspora spinosa containing a deleted spnK Shine-Dalgarno sequence is produced using a similar protocol as described in Example 2. Two fragments of at least 1,500 bp located upstream and downstream of the spnK Shine-Dalgarno sequence are PCR amplified. These fragments do not contain the following sequence 5′-AGGAGCTC-3′. The two fragments are ligated together within a plasmid such as pOJ260 which can be used for conjugation with Saccharopolyspora spinosa.

The desired plasmid is transformed into the E. coli conjugation donor strain ET12567/pUZ8002. A positively transformed strain is confirmed via restriction enzyme digestion. Upon confirmation that the E. coli strain containing the plasmid is correct, conjugation of the E. coli cells with Saccharopolyspora spinosa is performed according to the method described in Matsushima et al., (1994). Transfer of the plasmid from E. coli donor cells and the subsequent integration of the plasmid into the genome of Saccharopolyspora spinosa is selected for using resistance to an antibiotic.

Integration of the plasmid within the chromosome of Saccharopolyspora spinosa is molecularly characterized via PCR amplification of the specific genomic DNA region. Briefly, genomic DNA is isolated and the insertion containing the spnK Shine-Dalgarno sequence is PCR amplified, cloned, and sequenced. The sequencing data indicates that the spnK Shine-Dalgarno deletion construct integrates into the spnJK region via single crossover homologous recombination.

Double crossover mutants are obtained which contain the disrupted spnK Shine-Dalgarno sequence using the protocol described in Example 2. Colonies that are unable to grow on BHI agar plates containing the antibiotics which are selected for by the marker present on the vector backbone are identified as candidates of double crossover mutants and are selected for validation using PCR. Primers designed to bind within the spnK and spnJ gene are used. The resulting PCR product is sub-cloned into a plasmid using the TOPO® Cloning Technology (Invitrogen; Carlsbad Calif.). Bacterial colonies which contain the PCR product, cloned within the TOPO® vector are isolated and confirmed via restriction enzyme digestion. DNA sequencing of the positive plasmid clones is performed. The sequencing results indicate that the spnK Shine-Dalgarno nucleotide sequence is disrupted from the genome of Saccharopolyspora spinosa. Fermentation of the spnK Shine-Dalgarno mutant strains of Saccharopolyspora spinosa can be performed under conditions described by Burns et al., (WO 2003070908). Analysis of the fermentation broth for the presence of spinosyn factors can be carried out under conditions described by Baltz et al., (U.S. Pat. No. 6,143,526). To confirm the presence of the spinosyn factors in the supernatant, extracts of the fermentation broth are dried down in a SpeedVac overnight followed by partition of the residue between water and ether. The ether layer is dried by evaporation under N₂ stream. The sample is then dissolved in acetone-d₆ and transferred to an NMR tube for 1D proton NMR acquisition. The NMR results indicated a presence of an excess of J/L over A/D. The NMR profiles are compared to those of spinosyn standards. Fermentation of the strains which contain the deleted spnK Shine-Dalgarno sequence mutation produces a spinosyn mixture containing spinosyn J and L, as compared to the control Saccharopolyspora spinosa which produces a spinosyn mixture containing spinosyn A and D.

### Example 5

### Reduction of 3′-O-Methyltransferase Expression Via Anti-Sense RNA Down-Regulation of spnK

A plasmid is designed to produce asRNA (anti-sense RNA) complementary to the spnK coding sequence. The resulting down-regulation of spnK gene expression results in a reduction of spnK activity.

The spnK coding sequence is PCR amplified and is cloned into a plasmid such as pOJ260 for integration into the chromosome of Saccharopolyspora spinosa. Alternatively, the spnK coding sequence can be cloned into a plasmid that is stably maintained and replicated within the cytosol of Saccharopolyspora spinosa. The resulting plasmid is constructed to produce spnK asRNA by expressing the anti-sense strand of spnK using a strong constitutive bacterial promoter. This spnK asRNA plasmid is transformed into the E. coli conjugation donor strain ET12567/pUZ8002. A positively transformed strain is confirmed via restriction enzyme digestion. Upon confirmation that the E. coli strain containing the plasmid is correct, conjugation of the plasmid from the E. coli donor cells with Saccharopolyspora spinosa is performed according to the method described by Matsushima et al., (1994). The transfer of the spnK asRNA plasmid from E. coli into Saccharopolyspora spinosa is selected for using resistance to an antibiotic; the resistance of which is encoded for on the spnK asRNA plasmid. Genomic DNA is isolated from the transconjugants and is used as template for PCR amplification to confirm the existence of the plasmid.

Fermentation of the strains of Saccharopolyspora spinosa containing the spnK asRNA plasmid can be performed under conditions described by Burns et al., (WO 2003070908). Analysis of the fermentation broth for the presence of spinosyn factors can be carried out under conditions described by Baltz et al, (U.S. Pat. No. 6,143,526). To confirm the presence of the spinosyn factors in the supernatant, extracts of the fermentation broth are dried down in a SpeedVac overnight followed by partition of the residue between water and ether. The ether layer is dried by evaporation under N₂ stream. The sample is then dissolved in acetone-d₆ and transferred to an NMR tube for 1D proton NMR acquisition. The NMR results indicated a presence of an excess of J/L over A/D. The NMR profiles are compared to those of spinosyn standards. Fermentation of the strains which contain the spnK asRNA plasmid produces a spinosyn mixture containing spinosyn J and L, as compared to the control Saccharopolyspora spinosa which produces a spinosyn mixture containing spinosyn A and D.

### Example 6

### Generation of Additional spnK Deletion Mutations

### Example 6.1

### Construction of the spnK 5′ End Deletion Vector

Genomic DNA of a spinosyn A and D producing strain (Hopwood et al., 1985) is PCR amplified to produce two DNA fragments. The first amplified fragment is about 1,500 bp in length, and is located directly upstream of the ATG start codon. The second amplified fragment is about 1,500 bp in length, and is located directly downstream of spnK base pair 61. The PCR amplifications are completed using methods known to those skilled in the art. Oligonucleotide primers are synthesized to incorporate restriction enzyme binding sequences. The resulting PCR products are digested with restriction enzymes that cleave the binding sequences incorporated by the primers. The fragments are ligated together and then ligated into corresponding restriction sites of plasmid pOJ260. The resulting ligation product is cloned into E. coli competent cells. Colonies are selected and screened for the desired ligation product via restriction enzyme digestion and DNA sequence analysis. Positive clones are identified and a selected clone is used for subsequent 5′ end deletion of spnK in Saccharopolyspora spinosa. The resulting sequence of the deleted spnK gene fragment within plasmid pOJ260 is presented in Table 3. Therefore a spnK start codon deletion would include the sequence: (SEQ ID NO: 10).

TABLE 3
Nucleotide sequence alignment of the deleted 5′ end of spnK

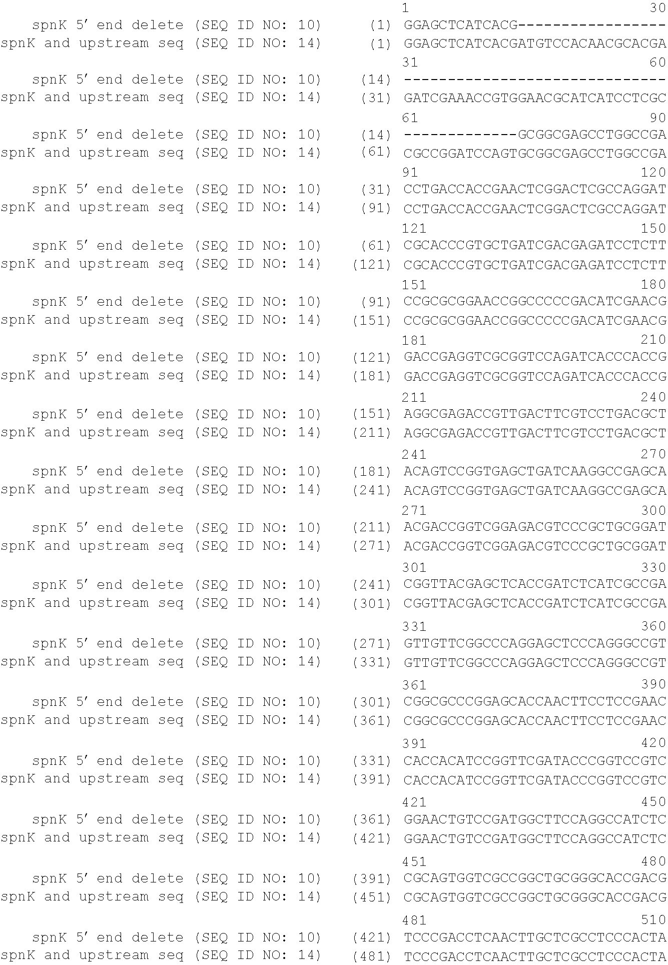

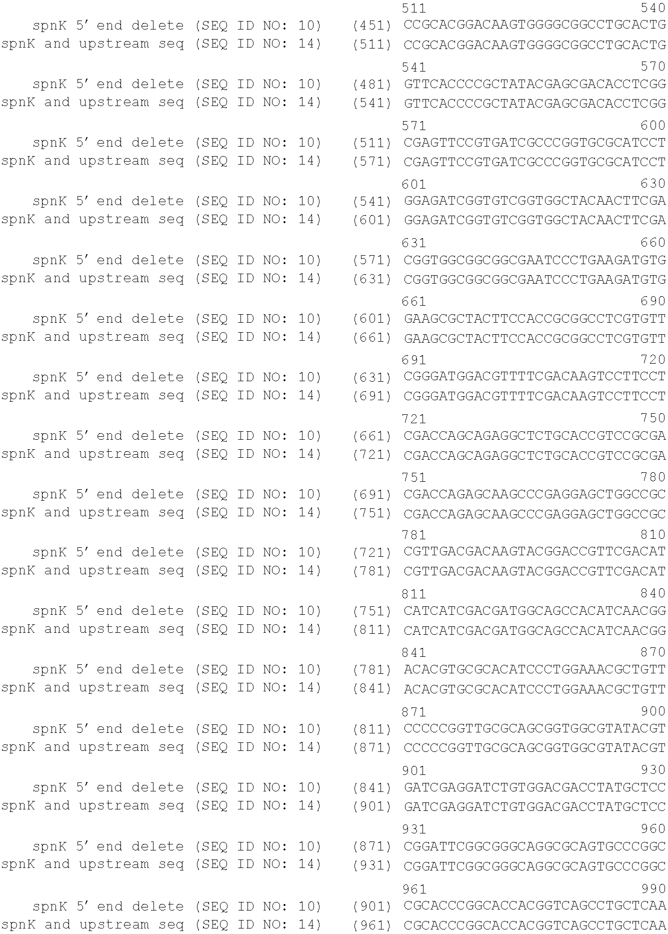

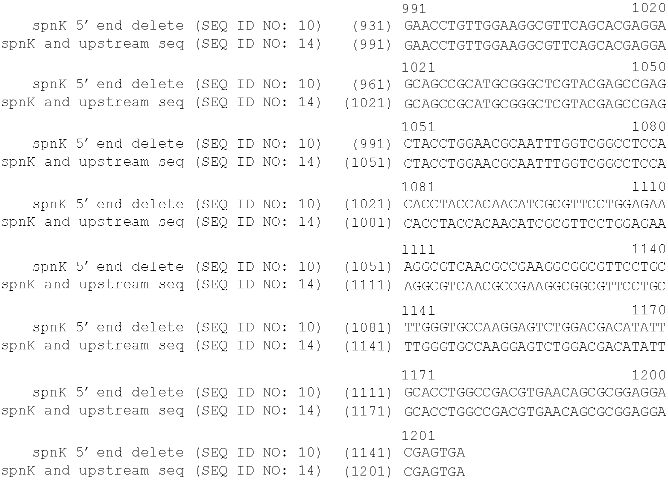

Conjugation of spnK Deletion Vector into Saccharopolyspora spinosa

Conjugation of the E. coli cells carrying the spnK 5′ end deletion construct with Saccharopolyspora spinosa is performed according to the method described in Matsushima et al., (1994) and exemplified in Example 2. Putative transconjugants resistant to apramycin, due to the presence of the apramycin resistance gene marker on the vector backbone of the spnK 5′ end deletion construct, are selected.

Confirmation of Transconjugants and Amplification of the spnK Region to Determine the Site of Integration

A single primary transconjugant grown on R6 media is transferred onto Brain Heart Infusion (BHI) agar plates supplemented with 50 μg/mL of apramycin and 25 μg/mL of nalidixic acid to confirm the resistance phenotype. Mycelia of the transconjugants are inoculated from the BHI plate into Tryptic Soy Broth (TSB) media supplemented with 50 μg/mL of apramycin. The culture is incubated at 29° C. with shaking at 250 rpm for 72 hours. Mycelia are harvested after 72 hours of incubation and genomic DNA is isolated. PCR is performed using the genomic DNA isolated from the transconjugant as template with primers designed to detect the single crossover mutant. The PCR amplification product results are sequenced. The sequencing data indicates that the spnK 5′ end deletion construct integrates into the spnJK region via single crossover homologous recombination.

Isolation of Double Crossover spnK 5′ End Deletion Mutant

A single crossover mutant resistant to apramycin is inoculated on BHI agar plates in the absence of apramycin and incubated at 29° C. for 14 days. Spores are harvested from the plates according to Hopwood et al., (1985) and stored in 20% glycerol at −80° C. Spores are inoculated onto new BHI agar plates without apramycin and plates are incubated at 29° C. for 14 days. This step is repeated multiple times. The spore preparation is diluted using 20% glycerol and the diluted spores are plated on BHI agar plates. Plates are incubated at 29° C. for 10 days for single colony development. Individual colonies are patched onto new BHI agar plates with and without apramycin. All plates are incubated at 29° C. for 10 days for mycelial development. Colonies that do not grow on BHI agar plates containing 50 μg/mL of apramycin are identified as candidates of double crossover mutants and are selected for validation using PCR.

### Identification and Validation of Double Crossover Mutants

Double crossover mutants are confirmed via PCR. Primers are designed to bind within the spnJ and spnK genes are used for PCR amplification. The sizes of the PCR products are determined via agarose gel electrophoresis. Double crossover mutants which result in a deletion of the 5′ end of the spnK gene are identified and selected based on the size of the PCR product. The size and DNA sequence of the PCR fragment indicates deletion of the ATG start codon and 5′ end of the spnK gene.

### Spinosyn Production Via Shake Flask Fermentation

Fermentation of the double crossover mutant can be performed under conditions described by Burns et al., (WO 2003070908). Analysis of the fermentation broth for the presence of spinosyn factors can be carried out under conditions described by Baltz et al., (U.S. Pat. No. 6,143,526). Fermentation of the double crossover mutant produces spinosyn J and L.

### Example 6.2

### Construction of the spnK In-Frame Deletion Vector

Two fragments of DNA are PCR amplified using genomic DNA of a spinosyn A and D producing strain (Hopwood et al., 1985). The first amplified fragment is about 1,500 bp in length, and is located directly upstream of the first putative S-adenosylmethionine-dependent methyltransferase domain. The second amplified fragment is about 1,500 bp in length, and is located directly downstream of the first putative S-adenosylmethionine-dependent methyltransferase domain. The PCR amplifications are completed using methods known to those skilled in the art. Oligonucleotide primers are synthesized to incorporate restriction enzyme binding sequences. The resulting PCR products are digested with restriction enzymes that cleave the binding sequences incorporated by the primers. The fragments are ligated together and then ligated into corresponding restriction sites of plasmid pOJ260. The resulting ligation product is cloned into E. coli competent cells. Colonies are selected and screened for the desired ligation product via restriction enzyme digestion and DNA sequence analysis. Positive clones are identified and a selected clone is used for subsequent in-frame deletion of the first putative S-adenosylmethionine-dependent methyltransferase domain of spnK within Saccharopolyspora spinosa. The resulting sequence of the deleted spnK gene fragment within plasmid pOJ260 is presented in Table 4. Therefore a spnK deletion would include the sequence: SEQ ID NO: 11.

TABLE 4
Nucleotide sequence alignment of the deleted first putative
S-adenosylmethionine-dependent methyltransferase domain of spnK
(putative S-adenosylmethionine-dependent
methyltransferase domains are underlined).

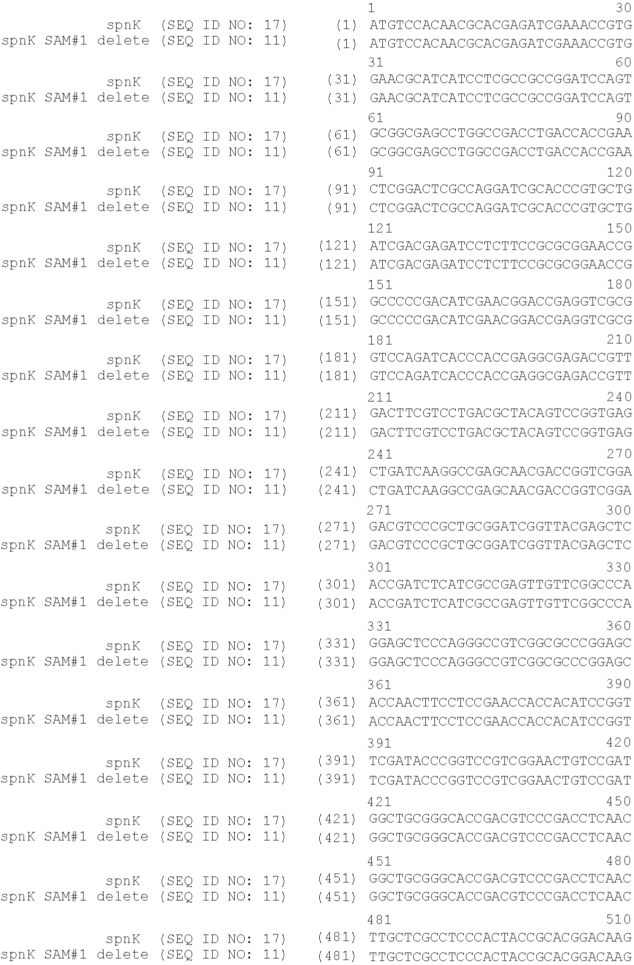

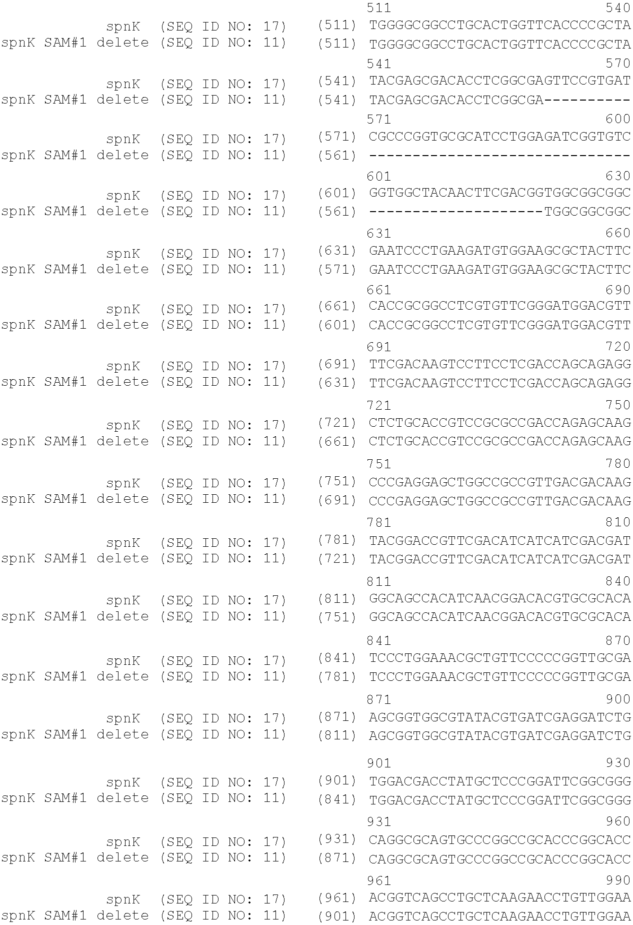

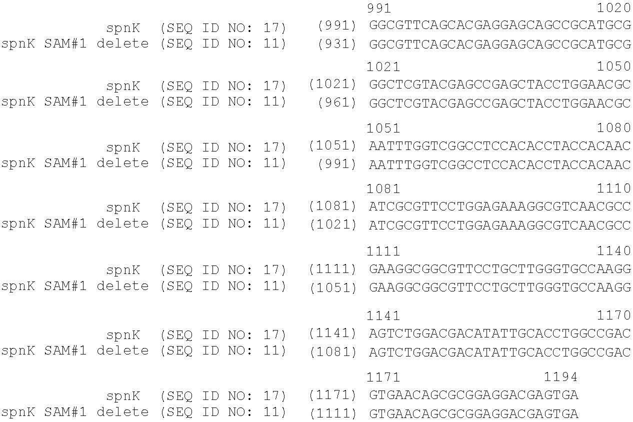

Conjugation of spnK Deletion Vector into Saccharopolyspora spinosa

Conjugation of the E. coli cells carrying the spnK in-frame deletion construct with Saccharopolyspora spinosa is performed according to the method described in Matsushima et al., (1994) and exemplified in Example 2. Putative transconjugants resistant to apramycin, due to the presence of the apramycin resistance gene marker on the vector backbone of the spnK in-frame deletion construct, are selected.

Confirmation of Transconjugants and Amplification of the spnK Region to Determine the Site of Integration

A single primary transconjugant grown on R6 media is transferred onto Brain Heart Infusion (BHI) agar plates supplemented with 50 μg/mL of apramycin and 25 μg/mL of nalidixic acid to confirm the resistance phenotype. Mycelia of the transconjugants are inoculated from the BHI plate into Tryptic Soy Broth (TSB) media supplemented with 50 μg/mL of apramycin. The culture is incubated at 29° C. with shaking at 250 rpm for 72 hours. Mycelia are harvested after 72 hours of incubation and genomic DNA is isolated. PCR is performed using the genomic DNA isolated from the transconjugant as template with primers designed to detect the single crossover mutant. The PCR amplification results are sequenced. The sequencing data indicates that the spnK in-frame deletion construct integrates into the spnK region via single crossover homologous recombination.

Isolation of Double Crossover spnK In-Frame Deletion Mutant

A single crossover mutant resistant to apramycin is inoculated on BHI agar plates in the absence of apramycin and incubated at 29° C. for 14 days. Spores are harvested from the plates according to Hopwood et al., (1985) and stored in 20% glycerol at −80° C. Spores are inoculated onto new BHI agar plates without apramycin and plates are incubated at 29° C. for 14 days. This step is repeated multiple times. The spore preparation is diluted using 20% glycerol and the diluted spores are plated on BHI agar plates. Plates are incubated at 29° C. for 10 days for single colony development. Individual colonies are patched onto new BHI agar plates with and without apramycin. All plates are incubated at 29° C. for 10 days for mycelial development. Colonies that do not grow on BHI agar plates containing 50 μg/mL of apramycin are identified as candidates of double crossover mutants and are selected for validation using PCR.

### Identification and Validation of Double Crossover Mutants

Double crossover mutants are confirmed via PCR. Primers are designed to bind within the spnK gene are used for PCR amplification. The sizes of the PCR products are determined via agarose gel electrophoresis. Double crossover mutants which result in a deletion of the first putative S-adenosylmethionine-dependent methyltransferase domain within the spnK gene are identified and selected based on the size of the PCR product. The size and DNA sequence of the PCR fragment indicates deletion of the first putative S-adenosylmethionine-dependent methyltransferase domain within the spnK gene.

### Spinosyn Production Via Shake Flask Fermentation

Fermentation of the double crossover mutant can be performed under conditions described by Burns et al., (WO 2003070908). Analysis of the fermentation broth for the presence of spinosyn factors can be carried out under conditions described by Baltz et al., (U.S. Pat. No. 6,143,526). Fermentation of the double crossover mutant produces spinosyn J and L.

### Example 6.3

### Construction of the spnK In-Frame Deletion Vector

Two fragments of DNA are PCR amplified using genomic DNA of a spinosyn A and D producing strain (Hopwood et al., 1985). The first amplified fragment is about 1,500 bp in length, and is located directly upstream of the second putative S-adenosylmethionine-dependent methyltransferase domain. The second amplified fragment is about 1,500 bp in length, and is located directly downstream of the second putative S-adenosylmethionine-dependent methyltransferase domain. The PCR amplifications are completed using methods known to those skilled in the art. Oligonucleotide primers are synthesized to incorporate restriction enzyme binding sequences. The resulting PCR products are digested with restriction enzymes that cleave the binding sequences incorporated by the primers. The fragments are ligated together and then ligated into corresponding restriction sites of plasmid pOJ260. The resulting ligation product is cloned into E. coli competent cells. Colonies are selected and screened for the desired ligation product via restriction enzyme digestion and DNA sequence analysis. Positive clones are identified and a selected clone is used for subsequent in-frame deletion of the second putative S-adenosylmethionine-dependent methyltransferase domain of spnK within Saccharopolyspora spinosa. The resulting sequence of the deleted spnK gene fragment within plasmid pOJ260 is presented in Table 5. Therefore a spnK deletion would include the sequence: SEQ ID NO: 12.

TABLE 5
Nucleotide sequence alignment of the deleted second putative
S-adenosylmethionine-dependent methyltransferase domain of spnK
(putative S-adenosylmethionine-dependent
methyltransferase domains are underlined).

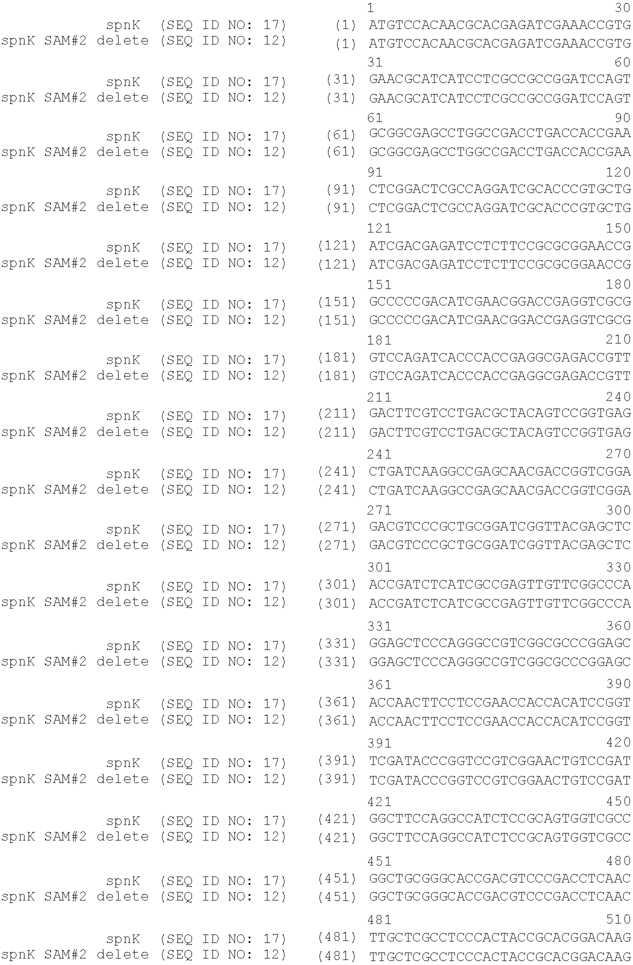

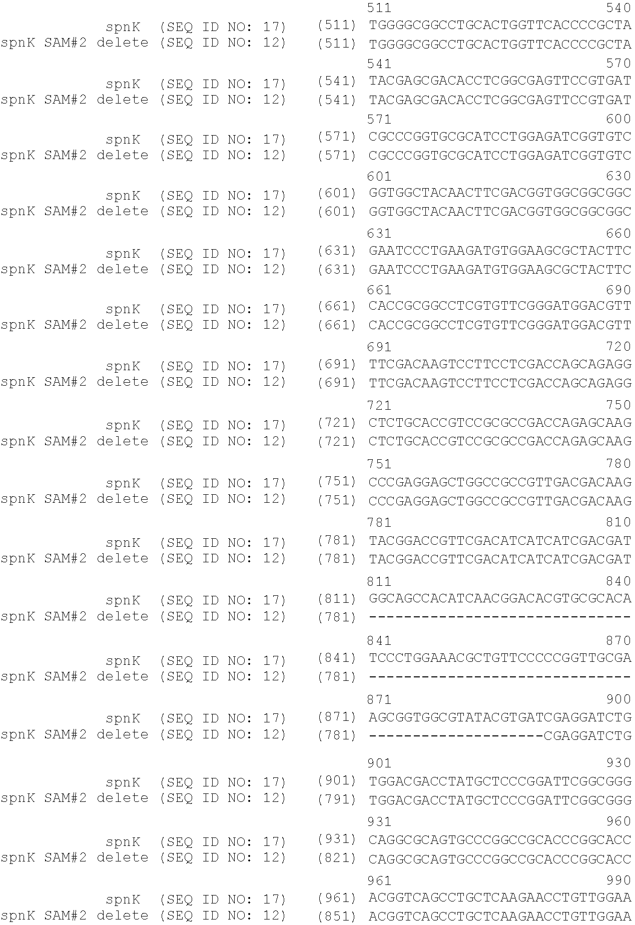

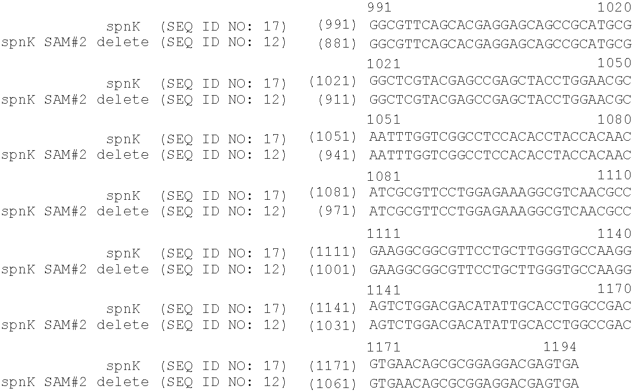

Conjugation of spnK Deletion Vector into Saccharopolyspora spinosa

Conjugation of the E. coli cells carrying the spnK in-frame deletion construct with Saccharopolyspora spinosa is performed according to the method described in Matsushima et al., (1994) and exemplified in Example 2. Putative transconjugants resistant to apramycin, due to the presence of the apramycin resistance gene marker on the vector backbone of the spnK in-frame deletion construct, are selected.

Confirmation of Transconjugants and Amplification of the spnK Region to Determine the Site of Integration

A single primary transconjugant grown on R6 media is transferred onto Brain Heart Infusion (BHI) agar plates supplemented with 50 μg/mL of apramycin and 25 μg/mL of nalidixic acid to confirm the resistance phenotype. Mycelia of the transconjugants are inoculated from the BHI plate into Tryptic Soy Broth (TSB) media supplemented with 50 μg/mL of apramycin. The culture is incubated at 29° C. with shaking at 250 rpm for 72 hours. Mycelia are harvested after 72 hours of incubation and genomic DNA is isolated. PCR is performed using the genomic DNA isolated from the transconjugant as template with primers designed to detect the single crossover mutant. The PCR amplification results are sequenced. The sequencing data indicates that the spnK in-frame deletion construct integrates into the spnK region via single crossover homologous recombination.

Isolation of Double Crossover spnK In-Frame Deletion Mutant

A single crossover mutant resistant to apramycin is inoculated on BHI agar plates in the absence of apramycin and incubated at 29° C. for 14 days. Spores are harvested from the plates according to Hopwood et al., (1985) and stored in 20% glycerol at −80° C. Spores are inoculated onto new BHI agar plates without apramycin and plates are incubated at 29° C. for 14 days. This step is repeated multiple times. The spore preparation is diluted using 20% glycerol and the diluted spores are plated on BHI agar plates. Plates are incubated at 29° C. for 10 days for single colony development. Individual colonies are patched onto new BHI agar plates with and without apramycin. All plates are incubated at 29° C. for 10 days for mycelial development. Colonies that do not grow on BHI agar plates containing 50 μg/mL of apramycin are identified as candidates of double crossover mutants and are selected for validation using PCR.

### Identification and Validation of Double Crossover Mutants

Double crossover mutants are confirmed via PCR. Primers are designed to bind within the spnK gene are used for PCR amplification. The sizes of the PCR products are determined via agarose gel electrophoresis. Double crossover mutants which result in a deletion of the second putative S-adenosylmethionine-dependent methyltransferase domain within the spnK gene are identified and selected based on the size of the PCR product. The size and DNA sequence of the PCR fragment indicates deletion of the second putative S-adenosylmethionine-dependent methyltransferase domain within the spnK gene.

### Spinosyn Production Via Shake Flask Fermentation

Fermentation of the double crossover mutant can be performed under conditions described by Burns et al., (WO 2003070908). Analysis of the fermentation broth for the presence of spinosyn factors can be carried out under conditions described by Baltz et al., (U.S. Pat. No. 6,143,526). Fermentation of the double crossover mutant produces spinosyn J and L.

### Example 6.4

### Construction of the spnK 3′ End Deletion Vector

Two fragments of DNA are PCR amplified using genomic DNA of a spinosyn A and D producing strain (Hopwood et al., 1985). The first amplified fragment is about 1,500 bp in length, and is located directly upstream of spnK base pair 1141. The second amplified fragment is about 1,500 bp in length, and is located directly downstream of the spnK termination codon and includes a portion of spnL. The PCR amplifications are completed using methods known to those skilled in the art. Oligonucleotide primers are synthesized to incorporate restriction enzyme binding sequences. The resulting PCR products are digested with restriction enzymes that cleave the binding sequences incorporated by the primers. The fragments are ligated together and then ligated into corresponding restriction sites of plasmid pOJ260. The resulting ligation product is cloned into E. coli competent cells. Colonies are selected and screened for the desired ligation product via restriction enzyme digestion and DNA sequence analysis. Positive clones are identified and a selected clone is used for subsequent deletion of the 3′ end of spnK within Saccharopolyspora spinosa. The resulting sequence of the deleted spnK gene fragment within plasmid pOJ260 is presented in Table 6. Therefore a spnK deletion would include the sequence: SEQ ID NO: 13.

TABLE 6
Nucleotide sequence alignment of the deleted 3′ end of spnK gene.

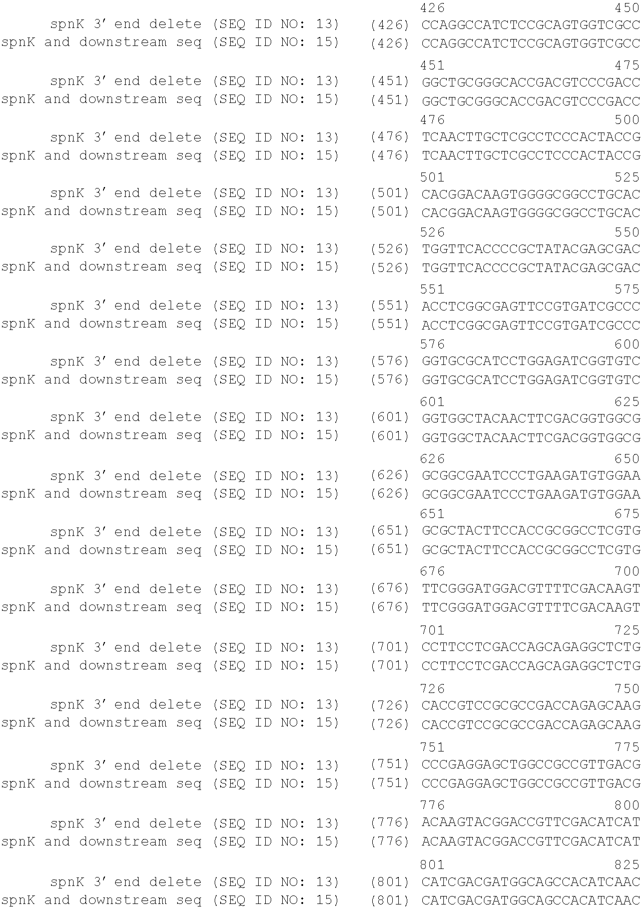

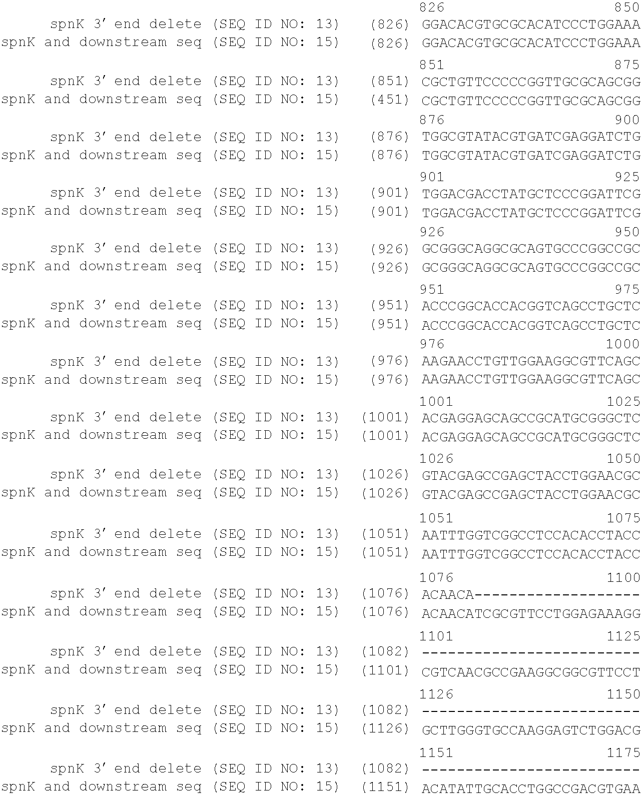

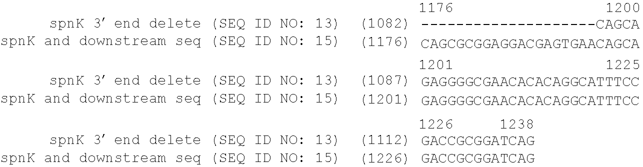

Conjugation of spnK Deletion Vector into Saccharopolyspora spinosa

Conjugation of the E. coli cells carrying the spnK 3′ end deletion construct with Saccharopolyspora spinosa is performed according to the method described in Matsushima et al., (1994) and exemplified in Example 2. Putative transconjugants resistant to apramycin, due to the presence of the apramycin resistance gene marker on the vector backbone of the spnK 3′ end deletion construct, are selected.

Confirmation of Transconjugants and Amplification of the spnK Region to Determine the Site of Integration

A single primary transconjugant grown on R6 media is transferred onto Brain Heart Infusion (BHI) agar plates supplemented with 50 μg/mL of apramycin and 25 μg/mL of nalidixic acid to confirm the resistance phenotype. Mycelia of the transconjugants are inoculated from the BHI plate into Tryptic Soy Broth (TSB) media supplemented with 50 μg/mL of apramycin. The culture is incubated at 29° C. with shaking at 250 rpm for 72 hours. Mycelia are harvested after 72 hours of incubation and genomic DNA is isolated. PCR is performed using the genomic DNA isolated from the transconjugant as template with primers designed to detect the single crossover mutant. The PCR amplification results are sequenced. The sequencing data indicates that the spnK 3′ end deletion construct integrates into the spnKL region via single crossover homologous recombination.

Isolation of Double Crossover spnK 3′ End Deletion Mutant

A single crossover mutant resistant to apramycin is inoculated on BHI agar plates in the absence of apramycin and incubated at 29° C. for 14 days. Spores are harvested from the plates according to Hopwood et al., (1985) and stored in 20% glycerol at −80° C. Spores are inoculated onto new BHI agar plates without apramycin and plates are incubated at 29° C. for 14 days. This step is repeated multiple times. The spore preparation is diluted using 20% glycerol and the diluted spores are plated on BHI agar plates. Plates are incubated at 29° C. for 10 days for single colony development. Individual colonies are patched onto new BHI agar plates with and without apramycin. All plates are incubated at 29° C. for 10 days for mycelial development. Colonies that do not grow on BHI agar plates containing 50 μg/mL of apramycin are identified as candidates of double crossover mutants and are selected for validation using PCR.

### Identification and Validation of Double Crossover Mutants

Double crossover mutants are confirmed via PCR. Primers are designed to bind within the spnK and spnL genes are used for PCR amplification. The sizes of the PCR products are determined via agarose gel electrophoresis. Double crossover mutants which result in a deletion of the 3′ end of the spnK gene are identified and selected based on the size of the PCR product. The size and DNA sequence of the PCR fragment indicates deletion of the 3′ end of the spnK gene.

### Spinosyn Production Via Shake Flask Fermentation

Fermentation of the double crossover mutant can be performed under conditions described by Burns et al., (WO 2003070908). Analysis of the fermentation broth for the presence of spinosyn factors can be carried out under conditions described by Baltz et al., (U.S. Pat. No. 6,143,526). Fermentation of the double crossover mutant produces spinosyn J and L.

All patents and publications referenced are incorporated by reference herein in their entirety. The foregoing is illustrative of the present invention, and is not to be construed as limiting thereof. The invention is defined by the following claims, with equivalents of the claims to be included therein.

What is claimed is:

1. A process for converting a spinosad producing strain to a spinetoram precursor producing strain comprising producing a modification in the spnK gene to eliminate 3′-O-methyltransferase activity.

2. The process according to claim 1, wherein the modification is selected from the group consisting of an in-frame deletion, a point mutation, a deletion, and an insertion.

3. The process according to claim 2, wherein the in-frame deletion is selected from the group consisting of an in-frame deletion of a 5′ end, an in-frame deletion of a 3′ end, and an in-frame deletion of a spnK coding region.

4. The process according to claim 2, wherein the deletion is a single or multiple nucleotide base deletion that disrupts the normal reading frame of the spnK gene.

5. The process according to claim 2, wherein the insertion is a single or multiple nucleotide base insertion that disrupts the normal reading frame of the spnK gene.

6. The process according to claim 2, wherein the point mutation results in an amino acid substitution in the active site or the substrate binding site of the spnK gene.

7. The process according to claim 2, wherein the point mutation occurs in the base pair location selected from the group consisting of location 528, 589, 602, 668, 721, 794, 862, 895, 908, 937 and 1131.

8. The process according to claim 2, wherein the point mutation results from chemical mutagenesis.

9. The process according to claim 1, wherein the spnK gene is disabled through use of antisense technology.

10. The process according to claim 1, wherein the modification occurs within the spnK coding region.

11. A genetically modified host cell that produces a spinetoram precursor, wherein the genetically modified host cell is a prokaryotic host cell that does not normally produce significant amount of spinetoram precursor comprising producing a modification in the spnK gene to eliminate 3′-O-methyltransferase activity.

12. A method of converting a spinosad producing strain to a spinetoram precursor producing strain comprising disabling a spnK gene while maintaining spinosyn J and L production.

13. The method according to claim 12, wherein the disabling of the spnK gene is selected from the group consisting of an in-frame deletion, a point mutation, a deletion, and an insertion.

14. The method according to claim 12, wherein the disabling of the spnK gene is caused by manipulating a ribosome binding site.

15. The method according to claim 14, wherein the ribosome binding site is a spnK Shine-Dalgarno sequence.

16. The method according to claim 12, wherein the disabling of the spnK gene is caused by manipulating a promoter of a spnK gene.

17. The method according to claim 16, the promoter is cotranscribed with a promoter for spnJ.

18. The method according to claim 13, wherein the in-frame deletion is selected from the group consisting of an in-frame deletion of a 5′ end, an in-frame deletion of a 3′ end, and an in-frame deletion of a spnK coding region.

19. The method according to claim 13, wherein the deletion is a single or multiple nucleotide base deletion that disrupts the normal reading frame of the spnK gene.

20. The method according to claim 13, wherein the point mutation results in an amino acid substitution in the active site or the substrate binding site of the spnK gene.
